(19) United States
(12) Patent Application Publication
(10) Pub. No.: US 2026/0217710 A1
(43) Pub. Date: Jul. 30, 2026

(54) COMPOSITIONS AND METHODS FOR MODULATING HAIR GROWTH

(71) Applicants: The Regents of the University of California, Oakland, CA (US); Pelage Pharmaceuticals, Inc., Los Angeles, CA (US)

(72) Inventors: Daniel L. Sun, Los Angeles, CA (US); Michael E. Jung, Los Angeles, CA (US); Daniel W. Gil, Los Angeles, CA (US)

(21) Appl. No.: 19/460,796

(22) Filed: Jan. 27, 2026

Related U.S. Application Data
(63) Continuation of application No. 18/013,803, filed on Jun. 29, 2021, now Pat. No. 12,559,490.
(60) Provisional application No. 63/046,629, filed on Jun. 30, 2020.

(51) Int. Cl. C07D 471/04

(52) U.S. Cl. CPC C07D 471/04

(57) ABSTRACT

The present disclosure relates to novel compounds that are capable of inhibiting the mitochondrial pyruvate carrier and promoting hair growth. The disclosure further relates to methods of promoting hair growth or treating conditions or disorders affecting hair growth, such as baldness or alopecia.

### CROSS-REFERENCE TO RELATED APPLICATIONS

This application is a continuation of U.S. patent application Ser. No. 18/013,803, filed Dec. 29, 2022, which is the § 371 National Stage of PCT/US21/39502, filed Jun. 29, 2021, which claims the benefit of U.S. Provisional Patent Application No. 63/046,629, filed Jun. 30, 2020, which is incorporated by reference herein in its entirety.

### BACKGROUND

Hair follicle stem cells (HFSCs) undergo successive rounds of quiescence (telogen) punctuated by brief periods of proliferation correlating with the start of the hair cycle (telogen-anagen transition). Proliferation or activation of HFSCs is well known to be a prerequisite for advancement of the hair cycle. Despite advances in treatment options, baldness and alopecia continue to be conditions that cannot be successfully treated in many individuals. Some of the existing treatments are inconvenient for users, others require surgical intervention or other invasive procedures. Additional therapies are needed.

### SUMMARY

Described herein are compounds of Formula 1:

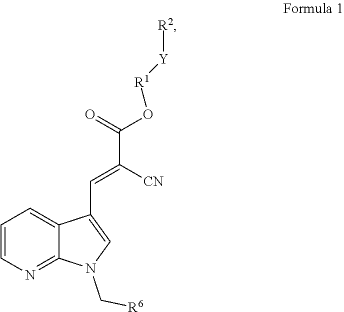

or a pharmaceutically acceptable salt thereof; wherein R¹ is —, —S(═O)₂—, an optionally substituted C₁₋₁₂ hydrocarbon group or an optionally substituted heterocycle; R² is H, an optionally substituted C₁₋₆ alkyl, an optionally substituted carbocycle, or an optionally substituted heterocycle; Y is —,

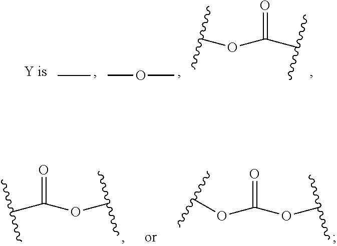

and R⁶ is

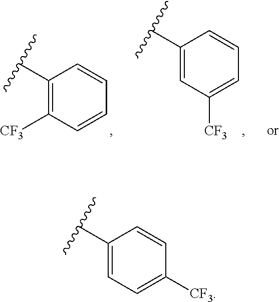 [SMILES: *c1ccccc1C(F)(F)F ; *c1cccc(C(F)(F)F)c1 ; *c1ccc(C(F)(F)F)cc1]

In other embodiments, R⁶ of Formula 1 is

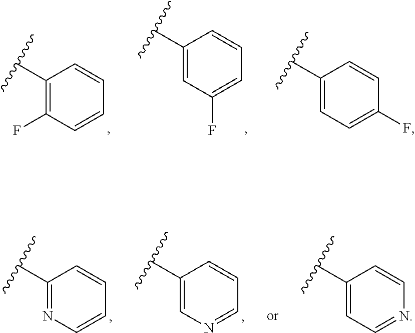 [SMILES: *c1ccccc1F ; *c1cccc(F)c1 ; *c1ccc(F)cc1 ; *c1ccccn1 ; *c1cccnc1 ; *c1ccncc1]

Some embodiments include a pharmaceutical composition comprising a compound described herein.

Some embodiments include a method of growing hair, comprising: administering a compound described herein to the skin of a mammal, including a human being, in the area where hair growth is intended.

Some embodiments include a method of growing hair comprising administering an MPO inhibitor to a mammal, including a human being, in need thereof. In some embodiments, the MPO inhibitor is a compound described herein.

Some embodiments include a method of treating a disorder affecting hair growth comprising administering a compound described herein to a mammal, including a human being, in need thereof. In some embodiments, the disorder is alopecia or baldness. Some embodiments include use of a compound described herein in the manufacture of a medicament for growing hair.

Some embodiments include a kit comprising a compound described herein and a label with instructions to administer the compound for a use described herein, such as growing hair.

### BRIEF DESCRIPTION OF THE DRAWINGS

FIG. 1 shows the conversion of ester prodrug (shown in gray) to the corresponding carboxylic acid API (shown in black) after 1 hour of incubation in homogenized human skin at 37° C. and pH 7.4.

FIG. 2 shows the conversion of ester prodrug (shown in gray) to the corresponding carboxylic acid API (shown in black) after 1 hour of incubation in homogenized human skin at 37° C. and pH 7.4.

### DETAILED DESCRIPTION

Described herein are compounds, compositions, and methods for modulating hair growth. Compounds of the present disclosure include substituted 7-azaindole compounds which may be useful for modulating hair growth.

Unless otherwise indicated, any reference to a compound herein by structure, name, or any other means, includes pharmaceutically acceptable salts, such as sodium, potassium, and ammonium salts; prodrugs, such as ester prodrugs; alternate solid forms, such as polymorphs, solvates, hydrates, etc.; deuterium-modified forms; Z and E olefin isomers; tautomers; or any other chemical species that may rapidly convert to a compound described herein under conditions in which the compounds are used as described herein. In some embodiments, the compound contains more than a natural abundance of deuterium. In some embodiments, one or more of the hydrogen atoms on the compound is replaced by deuterium so that the compound is at least 50%, at least 80%, at least 90%, at least 95%, or at least 99% deuterium in that position.

Unless otherwise indicated, when a compound or chemical structural feature (such as alkyl or aryl) is referred to as being “optionally substituted,” it includes a feature that has no substituents (i.e. unsubstituted), or a feature that is “substituted,” meaning that the feature has one or more substituents. The term “substituent” has the broadest meaning known to one of ordinary skill in the art, and includes a moiety that occupies a position normally occupied by one or more hydrogen atoms attached to a parent compound or structural feature. In some embodiments, a substituent may be an ordinary organic moiety known in the art, which may have a molecular weight (e.g. the sum of the atomic masses of the atoms of the substituent) of about 15 g/mol to about 50 g/mol, about 15 g/mol to about 100 g/mol, about 15 g/mol to about 150 g/mol, about 15 g/mol to about 200 g/mol, about 15 g/mol to about 300 g/mol, or about 15 g/mol to about 500 g/mol. In some embodiments, a substituent comprises, or consists of: 0-30, 0-20, 0-10, or 0-5 carbon atoms; and 0-30, 0-20, 0-10, or 0-5 heteroatoms, wherein each heteroatom may independently be: N, O, S, P, Si, F, Cl, Br, or I; provided that the substituent includes one C, N, O, S, P, Si, F, Cl, Br, or I atom.

Examples of substituents include, but are not limited to, compounds represented by an empirical formula: C₁₋₁₂H₃₋₂₉O₀₋₄N₀₋₄S₀₋₄F₀₋₂₅Cl₀₋₅Si₀₋₃P₀₋₃, C₀₋₁₂H₀₋₂₉O₁₋₄N₀₋₄S₀₋₄F₀₋₂₅Cl₀₋₅Si₀₋₃P₀₋₃, C₀₋₁₂H₀₋₂₉O₀₋₄N₁₋₄S₀₋₄F₀₋₂₅Cl₀₋₅Si₀₋₃P₀₋₃, C₀₋₁₂H₀₋₂₉O₀₋₄N₀₋₄S₁₋₄F₀₋₂₅Cl₀₋₅Si₀₋₃P₀₋₃, C₀₋₁₂H₀₋₂₉O₀₋₄N₀₋₄S₀₋₄F₁₋₂₅Cl₀₋₅Si₀₋₃P₀₋₃, C₀₋₁₂H₀₋₂₉O₀₋₄N₀₋₄S₀₋₄F₀₋₂₅Cl₁₋₅Si₀₋₃P₀₋₃, C₀₋₁₂H₀₋₂₉O₀₋₄N₀₋₄S₀₋₄F₀₋₂₅Cl₀₋₅Si₁₋₃P₀₋₃, C₀₋₁₂H₀₋₂₉O₀₋₄N₀₋₄S₀₋₄F₀₋₂₅Cl₀₋₅Si₀₋₃P₁₋₃, C₁₋₆H₃₋₁₆O₀₋₄N₀₋₄S₀₋₄F₀₋₁₃C₁₋₃Si₀₋₃P₀₋₃, C₀₋₆H₀₋₁₆O₁₋₄N₀₋₄S₀₋₄F₀₋₁₃Cl₀₋₃Si₀₋₃P₀₋₃, C₀₋₆H₀₋₁₇O₀₋₄N₁₋₄S₀₋₄F₀₋₁₃Cl₀₋₃Si₀₋₃P₀₋₃, C₀₋₆H₀₋₇O₀₋₄N₀₋₄S₁₋₄F₀₋₁₃Cl₀₋₃Si₀₋₃P₀₋₃, C₀₋₆H₀₋₁₇O₀₋₄N₀₋₄S₀₋₄F₁₋₁₃Cl₀₋₃Si₀₋₃P₀₋₃, C₀₋₆H₀₋₁₇O₀₋₄N₀₋₄S₀₋₄F₀₋₁₃C₁₋₁₃Si₀₋₃P₀₋₃, C₀₋₆H₀₋₁₇O₀₋₄N₀₋₄S₀₋₄F₀₋₁₃Cl₀₋₃Si₁₋₃P₀₋₃, or C₀₋₆H₀₋₁₇O₀₋₄N₀₋₄S₀₋₄F₀₋₁₃Cl₀₋₃Si₀₋₃P₁₋₃, C₁₋₁₂H₃₋₂₉O₀₋₄N₀₋₄S₀₋₄F₀₋₂₅Cl₀₋₅P₀₋₃, C₁₋₁₂H₃₋₂₇O₀₋₄N₀₋₂S₀₋₂F₀₋₂₅Cl₀₋₅P₀₋₁, C₁₋₁₂H₃₋₂₇O₀₋₄N₀₋₂, C₁₋₁₂H₃₋₂₅O₀₋₄, C₁₋₁₂H₃₋₂₇N₀₋₂, C₁₋₉H₃₋₂₁O₀₋₄N₀₋₂S₀₋₂F₀₋₁₉Cl₀₋₅P₀₋₁, C₁₋₉H₃₋₁₉F₀₋₁₉, C₁₋₉H₃₋₂₁O₀₋₄N₀₋₂, C₁₋₉H₃₋₁₉O₀₋₄, C₁₋₉H₃₋₂₁N₀₋₂, C₁₋₆H₃₁₋₅O₀₋₃N₀₋₂S₀₋₂F₀₁₋₃Cl₀₋₅P₀₋₁, C₁₋₆H₃₋₁₃F₀₋₁₃, C₁₋₆H₃₋₁₅O₀₋₄N₀₋₂, C₁₋₆H₃₋₁₃O₀₋₄, C₁₋₆H₃₋₁₅N₀₋₂, C₁₋₃H₃₋₉O₀₋₃N₀₋₂S₀₋₂F₀₋₁₃Cl₀₋₅P₀₋₁, C₁₋₃H₃₋₇F₀₋₇, C₁₋₃H₃₋₉O₀₋₃N₀₋₂, C₁₋₃H₃₋₇O₀₋₃, C₁₋₃H₃₋₉N₀₋₂, F, Cl, Br, I, OH, OR^{A}, SH, SR^{A}, NH₂, NHR^{A}, NR^{A}R^{B}, CF₃, CN, carboxylic acid, optionally substituted carboxylic ester, or optionally substituted C₁₋₆ alkyl, such as optionally substituted branched C₂₋₆ alkyl or optionally substituted linear C₁₋₆ alkyl, including optionally substituted branched or linear C₁₋₃ alkyl (e.g. —CH₃, —C₂H₅, —C₃H₇), optionally substituted branched, linear, or cyclic C₃₋₆ alkyl (e.g. —C₃H₇, —C₄H₉, —C₅H₁₁, —C₆H₁₃, cyclopropyl, cyclobutyl, cyclopentyl, cyclohexyl, etc.), alkenyl, alkynyl, heteroalkyl, heteroalkenyl, heteroalkynyl, aryl, heteroaryl, carbocycle, heterocycle, hydroxy, alkoxy, aryloxy, acyl, acyloxy, alkylcarboxylate, thiol, alkylthio, cyano, halo, thiocarbonyl, O-carbamyl, N-carbamyl, O-thiocarbamyl, N-thiocarbamyl, C-amido, N-amido, S-sulfonamido, N-sulfonamido, isocyanato, thiocyanato, isothiocyanato, nitro, silyl, sulfenyl, sulfinyl, sulfonyl, haloalkyl, haloalkoxyl, trihalomethanesulfonyl, trihalomethanesulfonamido, etc.

For convenience, the term “molecular weight” is used with respect to a moiety or part of a molecule to indicate the sum of the atomic masses of the atoms in the moiety or part of a molecule, even though it may not be a complete molecule.

As used herein, the term “alkyl” has the broadest meaning generally understood in the art, and may include a moiety composed of carbon and hydrogen containing no double or triple bonds. Alkyl may be linear alkyl, branched alkyl, cycloalkyl, or a combination thereof, and in some embodiments, may contain from one to thirty-five carbon atoms. In some embodiments, alkyl may include C₁₋₁₀ linear alkyl, such as methyl (—CH₃), ethyl (—CH₂CH₃), n-propyl (—CH₂CH₂CH₃), n-butyl (—CH₂CH₂CH₂CH₃), n-pentyl (—CH₂CH₂CH₂CH₂CH₃), n-hexyl (—CH₂CH₂CH₂CH₂CH₂CH₃), etc.; C₃₋₁₀ branched alkyl, such as C₃H₇ (e.g. iso-propyl), C₄H₉ (e.g. branched butyl isomers), C₅H₁₁ (e.g. branched pentyl isomers), C₆H₁₃ (e.g. branched hexyl isomers), C₇H₁₅ (e.g. branched heptyl isomers), etc.; C₃₋₁₀ cycloalkyl, such as C₃H₅ (e.g. cyclopropyl), C₄H₇ (e.g. cyclobutyl isomers such as cyclobutyl, methylcyclopropyl, etc.), C₅H₉ (e.g. cyclopentyl isomers such as cyclopentyl, methylcyclobutyl, dimethylcyclopropyl, etc.), C₆H₁₁ (e.g. cyclohexyl isomers), C₇H₁₃ (e.g. cycloheptyl isomers), etc.; and the like.

With respect to an optionally substituted moiety such as optionally substituted alkyl, a phrase such as “optionally substituted C₁₋₁₂ alkyl” refers to a C₁₋₁₂ alkyl that may be unsubstituted, or may have 1 or more substituents, and does not limit the number of carbon atoms in any substituent. A phrase such as “C₁₋₁₂ optionally substituted alkyl” refers to unsubstituted C₁₋₁₂ alkyl, or substituted alkyl wherein both the alkyl parent and all substituents have from 1-12 carbon atoms. Similar conventions may be applied to other optionally substituted moieties such as aryl and heteroaryl.

Empirical formulas, such as C₁₋₁₂H₃₋₂₅O₀₋₂N₀₋₂F₀₋₁₂, may be used to describe optionally substituted C₁₋₁₂ alkyl chemical compositions. In some embodiments, additional elements S, Si, P, other halogens, or other heteroatoms may also be included in the empirical formula.

The compounds described herein may have any of the following structural representations:

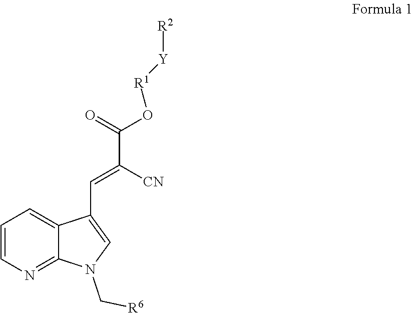

With respect to any relevant structural representation, such as Formula 1, R¹ is a bond (represented as —); —S(═O)₂—; an optionally substituted C₁₋₁₂ hydrocarbon group, including optionally substituted C₁₋₁₂ alkyl, such as optionally substituted branched C₂₋₁₂ alkyl or optionally substituted linear C₁₋₁₂ alkyl, including optionally substituted branched C₂₋₆ alkyl or linear C₁₋₆ alkyl, optionally substituted branched C₂₋₃ alkyl (e.g., —CH(CH₃)—, —CH(CH₂CH₃)—, —C(CH₃)₂—), or linear C₁₋₃ alkyl (e.g., —CH₂—, —C₂H₄—, —C₃H₆—), optionally substituted branched, linear, or cyclic C₃₋₆ alkyl (e.g. —C₃H₆—, —C₄H₈—, —C₅H₁₀—, —C₆H₁₂—, —CH(CH₂CH₃)—, —CH(CH₃)CH₂—, —C(CH₃)₂CH₂—, —CH₂CH(CH₃)CH₂—, —C(CH₃)(CH₂CH₃)—, —CH(CH₂CH₂CH₃)—, —C(CH₂CH₃)₂—, —C(CH₃)(CH₂CH₂CH₃)—, —CH(CH₂CH₂CH₂CH₃)—, —C(CH₃)(CH₂CH₂CH₂CH₃)—, —CH(CH₂CH₂CH₂CH₂CH₃)—, —C(CH₂CH₃)(CH₂CH₂CH₃)—, cyclopropyl, cyclobutyl, cyclopentyl, cyclohexyl, etc.), optionally substituted branched, linear, or cyclic C₆₋₉ alkyl (e.g., —C(CH₃)(CH₂CH₂CH₂CH₃)—, —CH(CH₂CH₂CH₂CH₂CH₃)—, —C(CH₂CH₃)(CH₂CH₂CH₃)—, —C₆H₁₂—, —C₇H₁₄—, —C₈H₁₆—, —C₉H₁₈—, cyclohexyl, cycloheptyl, cyclooctyl, cyclononyl, etc.), optionally substituted branched, linear, or cyclic C₉₋₁₂ alkyl, C₂₋₁₂ alkenyl, C₂₋₁₂ alkynyl, optionally substituted C₃₋₁₂ carbocycle, optionally substituted benzyl, etc.; optionally substituted carbocycle, including optionally substituted C₃₋₁₂ cycloalkyl, optionally substituted C₃₋₆ cycloalkyl, optionally substituted C₆₋₉ cycloalkyl, optionally substituted C₉₋₁₂ cycloalkyl, optionally substituted C₃₋₁₂ cycloalkenyl, optionally substituted C₃₋₆ cycloalkenyl, optionally substituted C₆₋₉ cycloalkenyl, optionally substituted C₉₋₁₂ cycloalkenyl, optionally substituted C₃₋₁₂ cycloalkynyl, optionally substituted C₃₋₆ cycloalkynyl, optionally substituted C₆₋₉ cycloalkynyl, optionally substituted C₉₋₁₂ cycloalkynyl, optionally substituted phenyl, optionally substituted naphthyl; or optionally substituted heterocycle, such as an optionally substituted monocyclic heterocycle having 3 ring carbon atoms and 1 ring oxygen atom, an optionally substituted monocyclic heterocycle having 4 ring carbon atoms and 1 ring oxygen atom, an optionally substituted monocyclic heterocycle having 5 ring carbon atoms and 1 ring oxygen atom, an optionally substituted monocyclic heterocycle having 6 ring carbon atoms and 1 ring oxygen atom, an optionally substituted monocyclic heterocycle having 7 ring carbon atoms and 1 ring oxygen atom, an optionally substituted monocyclic heterocycle having 3 ring carbon atoms and 1 ring sulfur atom, an optionally substituted monocyclic heterocycle having 4 ring carbon atoms and 1 ring sulfur atom, an optionally substituted monocyclic heterocycle having 5 ring carbon atoms and 1 ring sulfur atom, an optionally substituted monocyclic heterocycle having 6 ring carbon atoms and 1 ring sulfur atom, an optionally substituted monocyclic heterocycle having 7 ring carbon atoms and 1 ring sulfur atom, an optionally substituted monocyclic heterocycle having 3 ring carbon atoms and 1 ring nitrogen atom, an optionally substituted monocyclic heterocycle having 4 ring carbon atoms and 1 ring nitrogen atom, an optionally substituted monocyclic heterocycle having 5 ring carbon atoms and 1 ring nitrogen atom, an optionally substituted monocyclic heterocycle having 6 ring carbon atoms and 1 ring nitrogen atom, an optionally substituted monocyclic heterocycle having 7 ring carbon atoms and 1 ring nitrogen atom, an optionally substituted monocyclic heterocycle having 3 ring carbon atoms and 2 ring heteroatoms (N, O, and/or S), an optionally substituted monocyclic heterocycle having 4 ring carbon atoms and 2 ring heteroatoms (N, O, and/or S), an optionally substituted monocyclic heterocycle having 5 ring carbon atoms and 2 ring heteroatoms (N, O, and/or S), an optionally substituted monocyclic heterocycle having 6 ring carbon atoms and 2 ring heteroatoms (N, O, and/or S), an optionally substituted monocyclic heterocycle having 2 ring carbon atoms and 3 ring heteroatoms (N, O, and/or S), an optionally substituted monocyclic heterocycle having 3 ring carbon atoms and 3 ring heteroatoms (N, O, and/or S), an optionally substituted monocyclic heterocycle having 4 ring carbon atoms and 3 ring heteroatoms (N, O, and/or S), an optionally substituted monocyclic heterocycle having 5 ring carbon atoms and 3 ring heteroatoms (N, O, and/or S), an optionally substituted bicyclic heterocycle having 6 ring carbon atoms and 1 ring heteroatom (N, O, or S), an optionally substituted bicyclic heterocycle having 7 ring carbon atoms and 1 ring heteroatom (N, O, or S), an optionally substituted bicyclic heterocycle having 8 ring carbon atoms and 1 ring heteroatom (N, O, or S), an optionally substituted bicyclic heterocycle having 9 ring carbon atoms and 1 ring heteroatom (N, O, or S), an optionally substituted bicyclic heterocycle having 10 ring carbon atoms and 1 ring heteroatom (N, O, or S), an optionally substituted bicyclic heterocycle having 11 ring carbon atoms and 1 ring heteroatom (N, O, or S), an optionally substituted bicyclic heterocycle having 12 ring carbon atoms and 1 ring heteroatom (N, O, or S), an optionally substituted bicyclic heterocycle having 5 ring carbon atoms and 2 ring heteroatoms (N, O, and/or S), an optionally substituted bicyclic heterocycle having 6 ring carbon atoms and 2 ring heteroatoms (N, O, and/or S), an optionally substituted bicyclic heterocycle having 7 ring carbon atoms and 2 ring heteroatoms (N, O, and/or S), an optionally substituted bicyclic heterocycle having 8 ring carbon atoms and 2 ring heteroatoms (N, O, and/or S), an optionally substituted bicyclic heterocycle having 9 ring carbon atoms and 2 ring heteroatoms (N, O, and/or S), an optionally substituted bicyclic heterocycle having 10 ring carbon atoms and 2 ring heteroatoms (N, O, and/or S), an optionally substituted bicyclic heterocycle having 11 ring carbon atoms and 2 ring heteroatoms (N, O, and/or S), an optionally substituted bicyclic heterocycle having 5 ring carbon atoms and 3 ring heteroatoms (N, O, and/or S), an optionally substituted bicyclic heterocycle having 6 ring carbon atoms and 3 ring heteroatoms (N, O, and/or S), an optionally substituted bicyclic heterocycle having 7 ring carbon atoms and 3 ring heteroatoms (N, O, and/or S), an optionally substituted bicyclic heterocycle having 8 ring carbon atoms and 3 ring heteroatoms (N, O, and/or S), an optionally substituted bicyclic heterocycle having 9 ring carbon atoms and 3 ring heteroatoms (N, O, and/or S), an optionally substituted bicyclic heterocycle having 10 ring carbon atoms and 3 ring heteroatoms (N, O, and/or S), optionally substituted oxetane, optionally substituted tetrahydrofuran, optionally substituted dihydrofuran, optionally substituted furan, optionally substituted furanone, optionally substituted tetrahydropyran, optionally substituted dihydropyran, an optionally substituted pyran, optionally substituted tetrahydropyrone, optionally substituted dihydropyrone, optionally substituted pyrone, optionally substituted thietane, optionally substituted tetrahydrothiophene, optionally substituted dihydrothiophene, an optionally substituted thiophene, optionally substituted azetidine, optionally substituted pyrrolidine, optionally substituted pyrroline, optionally substituted pyrrole, optionally substituted piperidine, optionally substituted pyridine, optionally substituted oxazole, optionally substituted isoxazole, optionally substituted thiazole, optionally substituted isothiazole, optionally substituted pyrazolidine, optionally substituted imidazolidine, optionally substituted pyrazole, optionally substituted imidazole, optionally substituted tetrazole, optionally substituted sulfolane.

For the purposes of this disclosure, the term “alkyl” refers to both monovalent groups (such as —CH₃), bivalent groups (such as —CH₂—), or other hydrocarbon groups with higher valency that are free of double and triple bonds.

In some embodiments, R¹ is —. In some embodiments, R¹ is C₁₋₁₂ alkyl. In some embodiments, R¹ is linear C₁₋₁₂ alkyl. In some embodiments, R¹ is branched C₂₋₁₂ alkyl. In some embodiments, R¹ is —CH₂—, —C₂H₄—, —C₃H₆—, —C₃H₆—, —C₄H₈—, —C₅H₁₀—, —C₆H₁₂—, —C₇H₁₄—, —C₈H₁₆—, or —C₉H₁₈—. In some embodiments, R¹ is —CH₂—. In some embodiments, R¹ is —C₂H₄—. In some embodiments, R¹ is —C₃H₆—. In some embodiments, R¹ is —C₃H₆—. In some embodiments, R¹ is —C₄H₈—. In some embodiments, R¹ is —C₅H₁₀—. In some embodiments, R¹ is —C₆H₁₂—. In some embodiments, R¹ is —C₇H₁₄—. In some embodiments, R¹ is —C₈H₁₆—. In some embodiments, R¹ is —C₉H₁₈—. In some embodiments, R¹ is an optionally substituted linear C₁₋₁₂ alkyl. In some embodiments, R¹ is an optionally substituted branched C₂₋₁₂ alkyl. In some embodiments, R¹ is an optionally heteroatom substituted branched C₂₋₁₂ alkyl, such as a branched C₂₋₁₂ alkyl having polar substituents, including oxygen containing groups (e.g. —OH, ═O, OCH₃, etc.), sulfur containing groups (e.g. —SH, —SCH₃, SO₂, SO₃—, etc.), nitrogen containing groups (e.g. amino groups such as —NH₂, —NHCH₃, —N(CH₃)₂, quaternary ammonium salts such as —[N(CH₃)₂]*, —[N(CH₂CH₃)(CH₃)]*, —NO₂, —CN, etc.), fluorine containing groups (e.g. F, CF₃, CF₂CF₃, CHF₂, CH₂F, CF₂CF₂CF₃, etc.).

In some embodiments, R¹ is an optionally substituted carbocycle. In some embodiments, R¹ is optionally substituted cyclohexyl. In some embodiments, R¹ is an optionally substituted aryl. In some embodiments, R¹ is an optionally substituted phenyl. In some embodiments, R¹ is an optionally substituted benzyl. In some embodiments, R¹ is an optionally substituted heteroaryl. In some embodiments, R¹ is an optionally substituted heterocycle. In some embodiments wherein R¹ is an optionally substituted heterocycle, a carbon atom of the heterocycle (rather than a heteroatom of the heterocycle) is directly attached to O. In some embodiments wherein R¹ is an optionally substituted heterocycle, a carbon atom of the heterocycle ring (rather than a heteroatom of the heterocycle ring) is directly attached to Y. In some embodiments, R¹ is an optionally heteroatom substituted carbocycle, such as a carbocycle having polar substituents, including oxygen containing groups (e.g. —OH, ═O, OCH₃, etc.), sulfur containing groups (e.g. —SH, —SCH₃, SO₂, SO₃—, etc.), nitrogen containing groups (e.g. amino groups such as —NH₂, —NHCH₃, —N(CH₃)₂, quaternary ammonium salts such as —[N(CH₃)₂]*, —[N(CH₂CH₃)(CH₃)]*, —NO₂, —CN, etc.), fluorine containing groups (e.g. F, CF₃, CF₂CF₃, CHF₂, CH₂F, CF₂CF₂CF₃, etc.). In some embodiments, R¹ is an optionally heteroatom substituted heterocycle, such as a heterocycle having polar substituents, including oxygen containing groups (e.g. —OH, ═O, OCH₃, etc.), sulfur containing groups (e.g. —SH, —SCH₃, SO₂, SO₃, etc.), nitrogen containing groups (e.g. —NH₂, —NHCH₃, —N(CH₃)₂, —NO₂, —CN, etc.), fluorine containing groups (F, CF₃, CF₂CF₃, CHF₂, CH₂F, CF₂CF₂CF₃, etc.). In some embodiments, R¹ is an optionally heteroatom substituted benzyl, such as a benzyl having polar substituents, including oxygen containing groups (e.g. —OH, ═O, OCH₃, etc.), sulfur containing groups (e.g. —SH, —SCH₃, SO₂, SO₃⁻, etc.), nitrogen containing groups (e.g. —NH₂, —NHCH₃, —N(CH₃)₂, —NO₂, —CN, etc.), fluorine containing groups (e.g. F, CF₃, CF₂CF₃, CHF₂, CH₂F, CF₂CF₂CF₃, etc.). In some embodiments, R¹ is an optionally substituted oxetane. In some embodiments, R¹ is an optionally substituted oxetane having a carbon atom of the oxetane ring directly attached to the O atom.

In some embodiments, R¹ is an optionally substituted tetrahydrofuran. In some embodiments, R¹ is an optionally substituted tetrahydrofuran having a carbon atom of the tetrahydrofuran ring directly attached to the O atom.

In some embodiments, R¹ is an optionally substituted dihydrofuran. In some embodiments, R¹ is an optionally substituted dihydrofuran having a carbon atom of the dihydrofuran ring directly attached to the O atom.

In some embodiments, R¹ is an optionally substituted furan. In some embodiments, R¹ is an optionally substituted furan having a carbon atom of the furan ring directly attached to the O atom.

In some embodiments, R¹ is an optionally substituted furanone. In some embodiments, R¹ is an optionally substituted furanone having a carbon atom of the furanone ring directly attached to the O atom.

In some embodiments, R¹ is an optionally substituted tetrahydropyran. In some embodiments, R¹ is an optionally substituted tetrahydropyran having a carbon atom of the tetrahydropyran ring directly attached to the O atom.

In some embodiments, R¹ is an optionally substituted dihydropyran. In some embodiments, R¹ is an optionally substituted dihydropyran having a carbon atom of the dihydropyran ring directly attached to the O atom.

In some embodiments, R¹ is an optionally substituted pyran. In some embodiments, R¹ is an optionally substituted pyran having a carbon atom of the pyran ring directly attached to the O atom.

In some embodiments, R¹ is an optionally substituted tetrahydropyrone. In some embodiments, R¹ is an optionally substituted tetrahydropyrone having a carbon atom of the tetrahydropyrone ring directly attached to the O atom.

In some embodiments, R¹ is an optionally substituted dihydropyrone. In some embodiments, R¹ is an optionally substituted dihydropyrone having a carbon atom of the dihydropyrone ring directly attached to the O atom.

In some embodiments, R¹ is an optionally substituted pyrone. In some embodiments, R¹ is an optionally substituted pyrone having a carbon atom of the pyrone ring directly attached to the O atom.

In some embodiments, R¹ is an optionally substituted thietane. In some embodiments, R¹ is an optionally substituted thietane having a carbon atom of the thietane ring directly attached to the O atom.

In some embodiments, R¹ is an optionally substituted tetrahydrothiophene. In some embodiments, R¹ is an optionally substituted tetrahydrothiophene having a carbon atom of the tetrahydrothiophene ring directly attached to the O atom.

In some embodiments, R¹ is an optionally substituted dihydrothiophene. In some embodiments, R¹ is an optionally substituted dihydrothiophene having a carbon atom of the dihydrothiophene ring directly attached to the O atom.

In some embodiments, R¹ is an optionally substituted thiophene. In some embodiments, R¹ is an optionally substituted thiophene having a carbon atom of the thiophene ring directly attached to the O atom.

In some embodiments, R¹ is an optionally substituted azetidine. In some embodiments, R¹ is an optionally substituted azetidine having a carbon atom of the azetidine ring directly attached to the O atom. In some embodiments, R¹ is azetidine having an optionally substituted diphenylmethyl substituent. In some embodiments, R¹ is azetidine having an optionally substituted diphenylmethyl substituent attached to the nitrogen atom of the azetidine ring.

In some embodiments, R¹ is an optionally substituted pyrrolidine. In some embodiments, R¹ is an optionally substituted pyrrolidine having a carbon atom of the pyrrolidine ring directly attached to the O atom.

In some embodiments, R¹ is an optionally substituted pyrroline. In some embodiments, R¹ is an optionally substituted pyrroline having a carbon atom of the pyrroline ring directly attached to the O atom.

In some embodiments, R¹ is an optionally substituted pyrrole. In some embodiments, R¹ is an optionally substituted pyrrole having a carbon atom of the pyrrole ring directly attached to the O atom.

In some embodiments, R¹ is an optionally substituted piperidine. In some embodiments, R¹ is an optionally substituted piperidine having a carbon atom of the piperidine ring directly attached to the O atom.

In some embodiments, R¹ is an optionally substituted pyridine. In some embodiments, R¹ is an optionally substituted pyridine having a carbon atom of the pyridine ring directly attached to the O atom.

In some embodiments, R¹ is an optionally substituted oxazole. In some embodiments, R¹ is an optionally substituted oxazole having a carbon atom of the oxazole ring directly attached to the O atom.

In some embodiments, R¹ is an optionally substituted isoxazole. In some embodiments, R¹ is an optionally substituted isoxazole having a carbon atom of the isoxazole ring directly attached to the O atom.

In some embodiments, R¹ is an optionally substituted thiazole. In some embodiments, R¹ is an optionally substituted thiazole having a carbon atom of the thiazole ring directly attached to the O atom.

In some embodiments, R¹ is an optionally substituted isothiazole. In some embodiments, R¹ is an optionally substituted isothiazole having a carbon atom of the isothiazole ring directly attached to the O atom.

In some embodiments, R¹ is an optionally substituted pyrazolidine. In some embodiments, R¹ is an optionally substituted pyrazolidine having a carbon atom of the pyrazolidine ring directly attached to the O atom.

In some embodiments, R¹ is an optionally substituted imidazolidine. In some embodiments, R¹ is an optionally substituted imidazolidine having a carbon atom of the imidazolidine ring directly attached to the O atom.

In some embodiments, R¹ is an optionally substituted pyrazole. In some embodiments, R¹ is an optionally substituted pyrazole having a carbon atom of the pyrazole ring directly attached to the O atom.

In some embodiments, R¹ is an optionally substituted imidazole. In some embodiments, R¹ is an optionally substituted imidazole having a carbon atom of the imidazole ring directly attached to the O atom.

In some embodiments, R¹ is an optionally substituted tetrazole. In some embodiments, R¹ is an optionally substituted tetrazole having a carbon atom of the tetrazole ring directly attached to the O atom.

In some embodiments, R¹ is an optionally substituted sulfolane. In some embodiments, R¹ is an optionally substituted sulfolane having a carbon atom of the sulfolane ring directly attached to the O atom.

In some embodiments, R¹ is —S(═O)₂—.

In some embodiments, R¹ is —CH₂—, —CH₂CH(CH₃)CH₂—, or oxetane having a carbon atom of the oxetane ring directly attached to the O atom. In some embodiments, R¹ is —CH₂—. In some embodiments, R¹ is —CH₂CH(CH₃)CH₂—. In some embodiments, R¹ is oxetane having a carbon atom of the oxetane ring directly attached to the O atom.

In some embodiments, for a compound of Formula 1, R¹ is —, —CH₂—, an optionally substituted C₃₋₁₂ hydrocarbon group, or an optionally substituted heterocycle having a carbon atom directly attached to the O atom.

With respect to any relevant structural representation, such as Formula 1, R² is H; optionally substituted C₁₋₁₂ alkyl, optionally substituted C₁₋₆ alkyl group, such as optionally substituted branched C₃₋₆ alkyl or linear C₁₋₆ alkyl, optionally substituted branched C₃ alkyl (e.g., —CH(CH₃)₂), or optionally substituted linear C₁₋₃ alkyl (e.g., —CH₃, —C₂H₅, —C₃H₇), optionally substituted branched, linear, or cyclic C₃₋₆ alkyl (e.g. —C₃H₇, —C₄H₉, —C₅H₁₁, —C₆H₁₃, —CH(CH₃)₂, —CH(CH₃)(CH₂CH₃), —C(CH₃)₃, —CH(CH₂CH₃)₂, —CH(CH₃)(CH₂CH₂CH₃), —C(CH₃)₂(CH₂CH₃), —CH₂CH₂CH₂CH(CH₃)₂, —CH₂CH(CH₃)CH₂CH₂CH₃, —CH₂CH₂CH(CH₃)CH₂CH₃, —CH(CH₂CH₃)(CH₂CH₂CH₃), —C(CH₃)(CH₂CH₃)₂, cyclopropyl, cyclobutyl, cyclopentyl, cyclohexyl, etc.); optionally substituted carbocycle, including optionally substituted C₃₋₆ cycloalkyl, optionally substituted C₃₋₆ cycloalkenyl, optionally substituted C₃₋₆ cycloalkynyl, optionally substituted phenyl; or optionally substituted heterocycle such as an optionally substituted monocyclic heterocycle having 3 ring carbon atoms and 1 ring oxygen atom, an optionally substituted monocyclic heterocycle having 4 ring carbon atoms and 1 ring oxygen atom, an optionally substituted monocyclic heterocycle having 5 ring carbon atoms and 1 ring oxygen atom, an optionally substituted monocyclic heterocycle having 6 ring carbon atoms and 1 ring oxygen atom, an optionally substituted monocyclic heterocycle having 7 ring carbon atoms and 1 ring oxygen atom, an optionally substituted monocyclic heterocycle having 3 ring carbon atoms and 1 ring sulfur atom, an optionally substituted monocyclic heterocycle having 4 ring carbon atoms and 1 ring sulfur atom, an optionally substituted monocyclic heterocycle having 5 ring carbon atoms and 1 ring sulfur atom, an optionally substituted monocyclic heterocycle having 6 ring carbon atoms and 1 ring sulfur atom, an optionally substituted monocyclic heterocycle having 7 ring carbon atoms and 1 ring sulfur atom, an optionally substituted monocyclic heterocycle having 3 ring carbon atoms and 1 ring nitrogen atom, an optionally substituted monocyclic heterocycle having 4 ring carbon atoms and 1 ring nitrogen atom, an optionally substituted monocyclic heterocycle having 5 ring carbon atoms and 1 ring nitrogen atom, an optionally substituted monocyclic heterocycle having 6 ring carbon atoms and 1 ring nitrogen atom, an optionally substituted monocyclic heterocycle having 7 ring carbon atoms and 1 ring nitrogen atom, an optionally substituted monocyclic heterocycle having 3 ring carbon atoms and 2 ring heteroatoms (N, O, and/or S), an optionally substituted monocyclic heterocycle having 4 ring carbon atoms and 2 ring heteroatoms (N, O, and/or S), an optionally substituted monocyclic heterocycle having 5 ring carbon atoms and 2 ring heteroatoms (N, O, and/or S), an optionally substituted monocyclic heterocycle having 6 ring carbon atoms and 2 ring heteroatoms (N, O, and/or S), an optionally substituted monocyclic heterocycle having 2 ring carbon atoms and 3 ring heteroatoms (N, O, and/or S), an optionally substituted monocyclic heterocycle having 3 ring carbon atoms and 3 ring heteroatoms (N, O, and/or S), an optionally substituted monocyclic heterocycle having 4 ring carbon atoms and 3 ring heteroatoms (N, O, and/or S), an optionally substituted monocyclic heterocycle having 5 ring carbon atoms and 3 ring heteroatoms (N, O, and/or S), an optionally substituted bicyclic heterocycle having 6 ring carbon atoms and 1 ring heteroatom (N, O, or S), an optionally substituted bicyclic heterocycle having 7 ring carbon atoms and 1 ring heteroatom (N, O, or S), an optionally substituted bicyclic heterocycle having 8 ring carbon atoms and 1 ring heteroatom (N, O, or S), an optionally substituted bicyclic heterocycle having 9 ring carbon atoms and 1 ring heteroatom (N, O, or S), an optionally substituted bicyclic heterocycle having 10 ring carbon atoms and 1 ring heteroatom (N, O, or S), an optionally substituted bicyclic heterocycle having 11 ring carbon atoms and 1 ring heteroatom (N, O, or S), an optionally substituted bicyclic heterocycle having 12 ring carbon atoms and 1 ring heteroatom (N, O, or S), an optionally substituted bicyclic heterocycle having 5 ring carbon atoms and 2 ring heteroatoms (N, O, and/or S), an optionally substituted bicyclic heterocycle having 6 ring carbon atoms and 2 ring heteroatoms (N, O, and/or S), an optionally substituted bicyclic heterocycle having 7 ring carbon atoms and 2 ring heteroatoms (N, O, and/or S), an optionally substituted bicyclic heterocycle having 8 ring carbon atoms and 2 ring heteroatoms (N, O, and/or S), an optionally substituted bicyclic heterocycle having 9 ring carbon atoms and 2 ring heteroatoms (N, O, and/or S), an optionally substituted bicyclic heterocycle having 10 ring carbon atoms and 2 ring heteroatoms (N, O, and/or S), an optionally substituted bicyclic heterocycle having 11 ring carbon atoms and 2 ring heteroatoms (N, O, and/or S), an optionally substituted bicyclic heterocycle having 5 ring carbon atoms and 3 ring heteroatoms (N, O, and/or S), an optionally substituted bicyclic heterocycle having 6 ring carbon atoms and 3 ring heteroatoms (N, O, and/or S), an optionally substituted bicyclic heterocycle having 7 ring carbon atoms and 3 ring heteroatoms (N, O, and/or S), an optionally substituted bicyclic heterocycle having 8 ring carbon atoms and 3 ring heteroatoms (N, O, and/or S), an optionally substituted bicyclic heterocycle having 9 ring carbon atoms and 3 ring heteroatoms (N, O, and/or S), an optionally substituted bicyclic heterocycle having 10 ring carbon atoms and 3 ring heteroatoms (N, O, and/or S), optionally substituted oxetane, optionally substituted tetrahydrofuran, optionally substituted dihydrofuran, optionally substituted furan, optionally substituted furanone, optionally substituted tetrahydropyran, optionally substituted dihydropyran, an optionally substituted pyran, optionally substituted tetrahydropyrone, optionally substituted dihydropyrone, optionally substituted pyrone, optionally substituted thietane, optionally substituted tetrahydrothiophene, optionally substituted dihydrothiophene, an optionally substituted thiophene, optionally substituted azetidine, optionally substituted pyrrolidine, optionally substituted pyrroline, optionally substituted pyrrole, optionally substituted piperidine, optionally substituted pyridine, optionally substituted oxazole, optionally substituted isoxazole, optionally substituted thiazole, optionally substituted isothiazole, optionally substituted pyrazolidine, optionally substituted imidazolidine, optionally substituted pyrazole, optionally substituted imidazole, optionally substituted tetrazole, optionally substituted sulfolane.

In some embodiments, R² is H. In some embodiments, R² is C₁₋₆ alkyl. In some embodiments, R² is branched C₂₋₆ alkyl. In some embodiments, R² is —CH₃, —C₂H₅, —C₃H₇, —C₄H₉, —C₅H₁₁, or —C₆H₁₃. In some embodiments, R² is —CH₃. In some embodiments, R² is —C₂H₅. In some embodiments, R² is —C₃H₇. In some embodiments, R² is —C₄H₉. In some embodiments, R² is —C₅H₁₁. In some embodiments, R² is —C₆H₁₃. In some embodiments, R² is an optionally substituted linear C₁₋₆ alkyl. In some embodiments, R² is isopropyl. In some embodiments, R² is isobutyl. In some embodiments, R² is tert-butyl. In some embodiments, R² is fluoro substituted C₁₋₆ alkyl, including C₁₋₆ perfluoralkyl. In some embodiments, R² is fluoro substituted branched C₂₋₆ alkyl, such as branched C₂₋₆ perfluoroalkyl. In some embodiments, R² is —CF₃, —C₂F₅, —C₃F₇, —C₄F₉, —C₅F₁₁, or —C₆F₁₃. In some embodiments, R² is —CF₃. In some embodiments, R² is —C₂F₅. In some embodiments, R² is —C₃F₇. In some embodiments, R² is —C₄F₉. In some embodiments, R² is —C₅F₁₁. In some embodiments, R² is —C₆F₁₃. In some embodiments, R² is CF₃. In some embodiments, R² is CHF₂. In some embodiments, R² is CH₂F. In some embodiments, R² is CF₂CF₃. In some embodiments, R² is CF₂CF₂CF₃. In some embodiments, R² is fluoro substituted isopropyl, including perfluoroisopropyl. In some embodiments, R² is fluoro substituted isobutyl, including perfluoroisobutyl. In some embodiments, R² is fluoro substituted tert-butyl including perfluoro-tert-butyl.

In some embodiments, R² is an optionally substituted carbocycle. In some embodiments, R² is optionally substituted cyclohexyl. In some embodiments, R² is an optionally substituted aryl. In some embodiments, R² is an optionally substituted phenyl. In some embodiments, R² is optionally substituted benzyl. In some embodiments, R² is an optionally substituted heteroaryl. In some embodiments, R² is an optionally substituted heterocycle. In some embodiments wherein R² is an optionally substituted heterocycle, a carbon atom of the heterocycle (rather than a heteroatom of the heterocycle) is directly attached to Y.

In some embodiments, R² is an optionally substituted carbocycle, such as a carbocycle having electron-withdrawing substituents including acyl groups (e.g., —C(O)R, etc.) esters (e.g., —CO₂R, etc.), amides (e.g., —C(O)NR₂, etc.), imides (e.g., —C(O)NRC(O)R, etc.), cyano (—CN), sulfones (e.g., —SO₂R, etc.), sulfonamides (e.g., —SO₂NR₂), fluorine or fluorine containing groups (e.g., F, CF₃, CF₂CF₃, CHF₂, CH₂F, CF₂CF₂CF₃, etc.), and/or nitro (—NO₂). In some aspects, R² is an electron-deficient heterocyclic moiety.

In some embodiments, R² is an optionally substituted oxetane. In some embodiments, R² is an optionally substituted oxetane having a carbon atom of the oxetane ring directly attached to Y.

In some embodiments, R² is an optionally substituted tetrahydrofuran. In some embodiments, R² is an optionally substituted tetrahydrofuran having a carbon atom of the tetrahydrofuran ring directly attached to Y.

In some embodiments, R² is an optionally substituted dihydrofuran. In some embodiments, R² is an optionally substituted dihydrofuran having a carbon atom of the dihydrofuran ring directly attached to Y.

In some embodiments, R² is an optionally substituted furan. In some embodiments, R² is an optionally substituted furan having a carbon atom of the furan ring directly attached to Y.

In some embodiments, R² is an optionally substituted furanone. In some embodiments, R² is an optionally substituted furanone having a carbon atom of the furanone ring directly attached to Y.

In some embodiments, R² is an optionally substituted tetrahydropyran. In some embodiments, R² is an optionally substituted tetrahydropyran having a carbon atom of the tetrahydropyran ring directly attached to Y.

In some embodiments, R² is an optionally substituted dihydropyran. In some embodiments, R² is an optionally substituted dihydropyran having a carbon atom of the dihydropyran ring directly attached to Y.

In some embodiments, R² is an optionally substituted pyran. In some embodiments, R² is an optionally substituted pyran having a carbon atom of the pyran ring directly attached to Y.

In some embodiments, R² is an optionally substituted tetrahydropyrone. In some embodiments, R² is an optionally substituted tetrahydropyrone having a carbon atom of the tetrahydropyrone ring directly attached to Y.

In some embodiments, R² is an optionally substituted dihydropyrone. In some embodiments, R² is an optionally substituted dihydropyrone having a carbon atom of the dihydropyrone ring directly attached to Y.

In some embodiments, R² is an optionally substituted pyrone. In some embodiments, R² is an optionally substituted pyrone having a carbon atom of the pyrone ring directly attached to Y.

In some embodiments, R² is an optionally substituted thietane. In some embodiments, R² is an optionally substituted thietane having a carbon atom of the thietane ring directly attached to Y.

In some embodiments, R² is an optionally substituted tetrahydrothiophene. In some embodiments, R² is an optionally substituted tetrahydrothiophene having a carbon atom of the tetrahydrothiophene ring directly attached to Y.

In some embodiments, R² is an optionally substituted dihydrothiophene. In some embodiments, R² is an optionally substituted dihydrothiophene having a carbon atom of the dihydrothiophene ring directly attached to Y.

In some embodiments, R² is an optionally substituted thiophene. In some embodiments, R² is an optionally substituted thiophene having a carbon atom of the thiophene ring directly attached to Y.

In some embodiments, R² is an optionally substituted azetidine. In some embodiments, R² is an optionally substituted azetidine having a carbon atom of the azetidine ring directly attached to Y. In some embodiments, R² is azetidine having an optionally substituted diphenylmethyl substituent. In some embodiments, R² is azetidine having an optionally substituted diphenylmethyl substituent attached to the nitrogen atom of the azetidine ring.

In some embodiments, R² is an optionally substituted pyrrolidine. In some embodiments, R² is an optionally substituted pyrrolidine having a carbon atom of the pyrrolidine ring directly attached to Y.

In some embodiments, R² is an optionally substituted pyrroline. In some embodiments, R² is an optionally substituted pyrroline having a carbon atom of the pyrroline ring directly attached to Y.

In some embodiments, R² is an optionally substituted pyrrole. In some embodiments, R² is an optionally substituted pyrrole having a carbon atom of the pyrrole ring directly attached to Y.

In some embodiments, R² is an optionally substituted piperidine. In some embodiments, R² is an optionally substituted piperidine having a carbon atom of the piperidine ring directly attached to Y.

In some embodiments, R² is an optionally substituted pyridine. In some embodiments, R² is an optionally substituted pyridine having a carbon atom of the pyridine ring directly attached to Y.

In some embodiments, R² is an optionally substituted oxazole. In some embodiments, R² is an optionally substituted oxazole having a carbon atom of the oxazole ring directly attached to Y.

In some embodiments, R² is an optionally substituted isoxazole. In some embodiments, R² is an optionally substituted isoxazole having a carbon atom of the isoxazole ring directly attached to Y.

In some embodiments, R² is an optionally substituted thiazole. In some embodiments, R² is an optionally substituted thiazole having a carbon atom of the thiazole ring directly attached to Y.

In some embodiments, R² is an optionally substituted isothiazole. In some embodiments, R² is an optionally substituted isothiazole having a carbon atom of the isothiazole ring directly attached to Y.

In some embodiments, R² is an optionally substituted pyrazolidine. In some embodiments, R² is an optionally substituted pyrazolidine having a carbon atom of the pyrazolidine ring directly attached to Y.

In some embodiments, R² is an optionally substituted imidazolidine. In some embodiments, R² is an optionally substituted imidazolidine having a carbon atom of the imidazolidine ring directly attached to Y.

In some embodiments, R² is an optionally substituted pyrazole. In some embodiments, R² is an optionally substituted pyrazole having a carbon atom of the pyrazole ring directly attached to Y.

In some embodiments, R² is an optionally substituted imidazole. In some embodiments, R² is an optionally substituted imidazole having a carbon atom of the imidazole ring directly attached to Y.

In some embodiments, R² is an optionally substituted tetrazole. In some embodiments, R² is an optionally substituted tetrazole having a carbon atom of the tetrazole ring directly attached to Y.

In some embodiments, R² is an optionally substituted sulfolane. In some embodiments, R² is an optionally substituted sulfolane having a carbon atom of the sulfolane ring directly attached to Y.

In some more particular but non-limiting forms, R² is H, —CH₃, —CH₂CH₃, —CH(CH₃)₂, or —C(CH₃)₃. In some embodiments, R² is —CH₂CH₃. In some embodiments, R² is —CH(CH₃)₂.

With respect to any relevant structural representation, such as Formula 1, in some embodiments, R² is CH₃ or C₃₋₁₂ alkyl, such as branched C₃ alkyl (e.g., —CH(CH₃)₂), or linear C₁₋₃ alkyl (e.g., —CH₃, —C₂H₅, —C₃H₇), branched, linear, or cyclic C₃₋₆ alkyl (e.g. —C₃H₇, —C₄H₉, —C₅H₁₁, —C₆H₁₃, —CH(CH₃)₂, —CH(CH₃)(CH₂CH₃), —C(CH₃)₃, —CH(CH₂CH₃)₂, —CH(CH₃)(CH₂CH₂CH₃), —C(CH₃)₂(CH₂CH₃), —CH₂CH₂CH₂CH(CH₃)₂, —CH₂CH(CH₃)CH₂CH₂CH₃, —CH₂CH₂CH(CH₃)CH₂CH₃, —CH(CH₂CH₃)(CH₂CH₂CH₃), —C(CH₃)(CH₂CH₃)₂, cyclopropyl, cyclobutyl, cyclopentyl, cyclohexyl, etc.).

With respect to any relevant structural representation, such as Formula 1, Y is —,

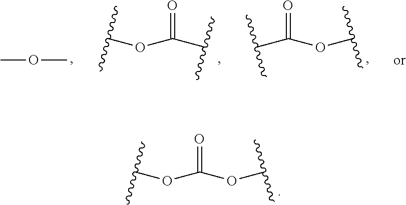

In some embodiments, Y is —. In some embodiments, Y is —O—. In some embodiments, Y is

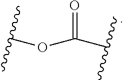 [SMILES: *OC(*)=O]

In some embodiments, Y is

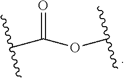 [SMILES: *OC(*)=O]

In some embodiments, Y is

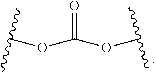 [SMILES: *OC(=O)O*]

In some embodiments, Y is — or

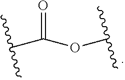 [SMILES: *OC(*)=O]

With respect to any relevant structural representation, such as Formula 1, R⁶ is

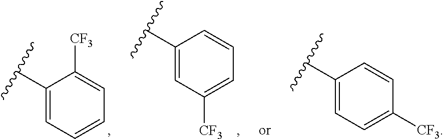 [SMILES: *c1ccccc1C(F)(F)F ; *c1cccc(C(F)(F)F)c1 ; *c1ccc(C(F)(F)F)cc1]

In other embodiments, R⁶ is

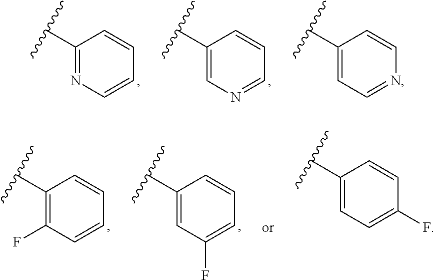 [SMILES: *c1ccccn1 ; *c1cccnc1 ; *c1ccncc1 ; *c1ccccc1F ; *c1cccc(F)c1 ; *c1ccc(F)cc1]

In some embodiments, R⁶ is

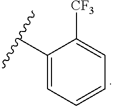 [SMILES: *c1ccccc1C(F)(F)F]

In some embodiments, R⁶ is

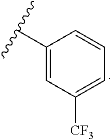 [SMILES: *c1cccc(C(F)(F)F)c1]

In some embodiments, R⁶ is

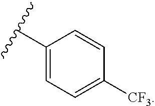 [SMILES: *c1ccc(C(F)(F)F)cc1]

is

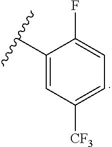 [SMILES: *c1cc(C(F)(F)F)ccc1F]

In some embodiments, R⁶ is

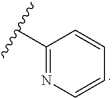 [SMILES: *c1ccccn1]

In some embodiments, R⁶ is

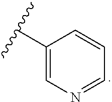 [SMILES: *c1cccnc1]

In some embodiments, R⁶ is

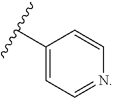 [SMILES: *c1ccncc1]

In some embodiments, R⁶ is

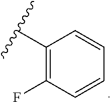 [SMILES: *c1ccccc1F]

In some embodiments, R⁶ is

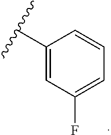 [SMILES: *c1cccc(F)c1]

In some embodiments, R⁶ is

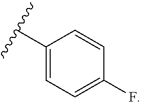 [SMILES: *c1ccc(F)cc1]

In some embodiments, the compound is a compound shown below, each of which may be optionally substituted:

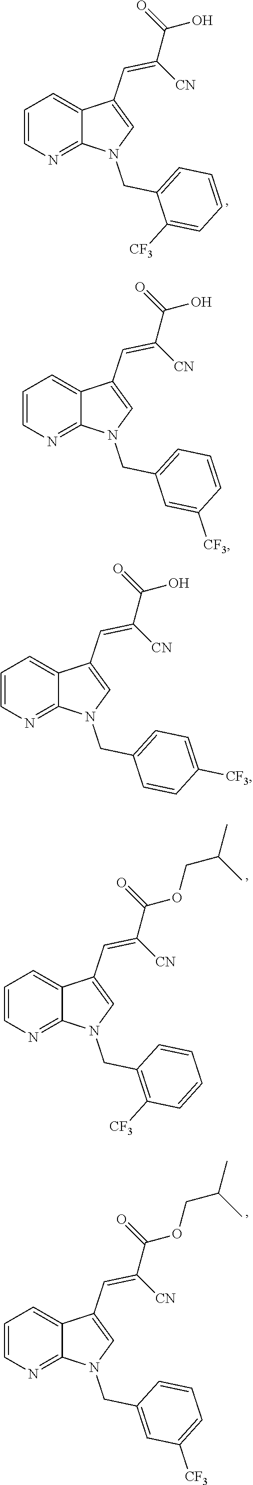

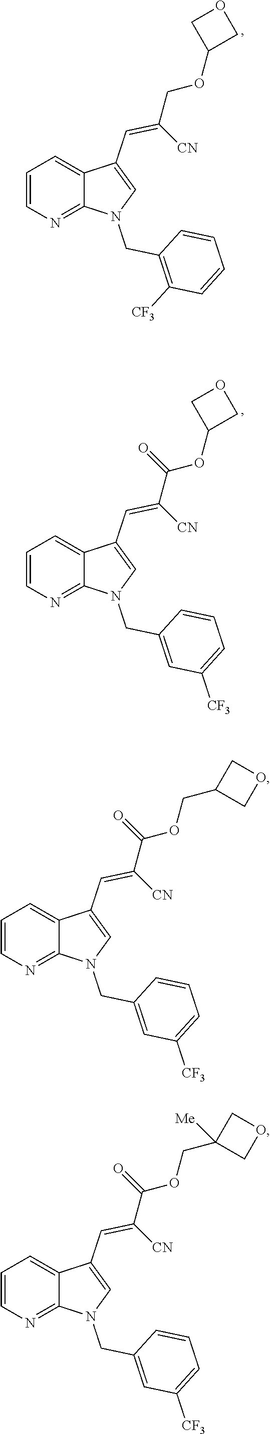

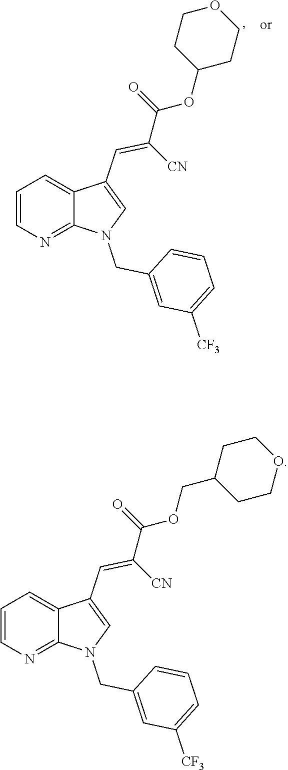 [SMILES: N#C/C(=C\c1cn(Cc2cccc(C(F)(F)F)c2)c2ncccc12)C(=O)OC1CCOCC1 ; N#C/C(=C\c1cn(Cc2cccc(C(F)(F)F)c2)c2ncccc12)C(=O)OCC1CCOCC1]

In other embodiments, the compound is a compound shown below, each of which may be optionally substituted:

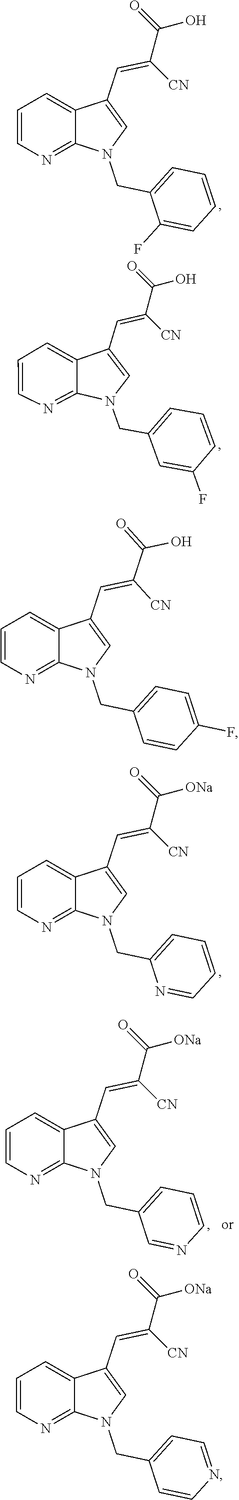

The compound described herein are useful for growing hair. For example, a compound described herein may be administered to the skin of a mammal in the area where hair growth is intended.

For use in growing hair, a compound described herein may be mixed with a dermatologically compatible vehicle or carrier, e.g. so that the compound is present at an amount of about 0.001-10% or about 0.01-2%. The vehicle which may be employed for a topical dermatological composition may comprise, for example, aqueous solutions such as e.g., physiological salines, oil, solutions, ointments, gels, creams, sprays, etc. In some embodiments, the vehicle may contain a solvent such as ethanol or polyethylene glycol. In some embodiments, the vehicle may also contain a penetration enhancer, e.g. to enhance penetration into the skin, such as transcutol P. The vehicle furthermore may contain dermatologically compatible preservatives such as e.g., benzalkonium chloride, surfactants like e.g., polysorbate 80, liposomes or polymers, for example, methyl cellulose, polyvinyl alcohol, polyvinyl pyrrolidone and hyaluronic acid; these may be used for increasing the viscosity.

Solubility may be important for certain formulations, such as topical formulations, since it may be that more soluble compounds enable delivery of higher concentrations of drug to the target tissues of skin. The solubility of a compound may depend upon the type of formulation. For example, a compound with higher aqueous solubility or polarity may provide higher concentrations of a drug to target tissues when applied with an aqueous, water-soluble, or polar formulation. On the other hand, a compound with higher lipid solubility may provide higher concentrations of a drug to target tissues when applied with an oil-based formulation.

In certain aspects, the compounds of the present disclosure are mitochondrial pyruvate oxidation (MPO) inhibitors. In some embodiments, the compounds described herein may inhibit mitochondrial pyruvate carrier (MPC). In certain embodiments, the MPO inhibitor is an MPC inhibitor. In some aspects, inhibiting MPO in a cell has the effect of enhancing lactate production in a cell and/or enhancing the activity of lactic acid dehydrogenase (LDH) in a cell, and promoting hair growth. In certain aspects, the present disclosure provides methods of promoting hair growth or treating a hair growth condition or disorder such as baldness or alopecia, comprising administering to a patient an MPO inhibitor (e.g., topically, such as with a pharmaceutical composition formulated for topical application), such as a compound of the present disclosure. In certain embodiments, the present disclosure provides methods of promoting hair growth or treating a hair growth condition or disorder such as baldness or alopecia, comprising administering to a patient an MPC inhibitor (e.g., topically, such as with a pharmaceutical composition formulated for topical application), such as a compound of the present disclosure. In some embodiments, inhibiting the MPO or the MPC in a cell has the effect of enhancing lactate production and/or enhancing the activity of LDH in a cell, and promoting hair growth.

For the purposes of this disclosure, the term “treat,” “treating,” or a similar term (such as “modulating”), includes cure, mitigation, treatment, or prevention of disease in man or other animals, or any other effect that would be associated with a “drug” as defined under 21 USC 321(g).

In certain aspects, the compounds of the present disclosure may be ester prodrugs. In other aspects, the compounds described herein may be thioester or amide prodrugs. In some embodiments, the compounds herein may show a higher rate of hydrolysis (such as a rate that is at least about 1.1 times higher, at least about 1.5 times higher, at least about 2 times higher, at least about 5 times higher, at least about 10 times higher, at least about 50 times higher, at least about 100 times higher, at least about 500 times higher, at least about 1,000 times higher, at least about 10,000 times higher, about 1.1-2 times higher, about 2-4 times higher, about 4-6 times higher, about 6-8 times higher, about 8-10 times higher, about 1.1-10 times higher, about 10-100 times higher, about 100-1,000 times higher, or about 1,000-10,000 times higher) relative to conventional alkyl (ethyl or methyl) esters. In some aspects, the compounds of the present disclosure may achieve a high level of hydrolyzed drug (carboxylic acid) in skin homogenate assays.

It is understood that topical delivery of an active pharmaceutical ingredient (API) for dermal indications comprises a balance of lipophilic and hydrophilic properties. It is believed that a compound having lipophilicity as a prodrug and hydrophilicity as the corresponding free acid API may achieve the goal of reaching the desired skin layer target (e.g., a hair follicle). The rate of hydrolysis of the prodrug in the layers of the skin may be adjusted to achieve the desired result.

In some embodiments, the compounds of the present disclosure undergo hydrolysis to release the active free carboxylic acid. In some aspects, the compounds of the present disclosure undergo hydrolysis to release the active free carboxylic acid at a rate that is enhanced relative to conventional prodrugs (e.g., JXL082). In some embodiments, the rate of hydrolysis may benefit the delivery of active pharmaceutical agent to potentiate hair growth.

In some aspects, the prodrug compounds of the present disclosure undergo hydrolysis in human skin homogenate faster than known prodrugs such as JXL082. In some embodiments, the concentration of carboxylic acid (API) released by a prodrug of the present disclosure is at least about 150% greater to about 20000% greater than the amount released by a conventional prodrug such as JXL082. In some embodiments, the concentration of carboxylic acid (API) released by a prodrug of the present disclosure is at least about 150-300% greater, about 300-500% greater, about 500-1000% greater, about 1000-2000% greater, about 2000-4000% greater, about 4000-7000% greater, about 7000-10000% greater, about 10000-15000% greater, or about 15000-20000% greater, or about any value in a range bounded by any of these ranges, than the amount released by a conventional prodrug such as JXL082.

In other embodiments, the compounds of the present disclosure may enhance hair growth in their free acid form.

### EXAMPLES

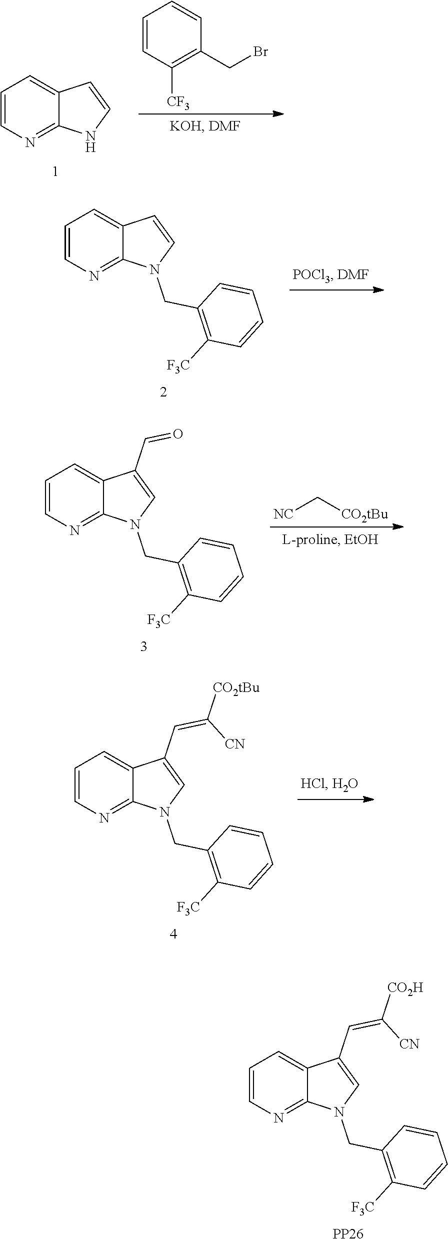

### Example 1: (E)-2-Cyano-3-(1-(2-(trifluoromethyl)benzyl)-1H-pyrrolo[2,3-b]pyridin-3-yl)acrylic acid (PP26)

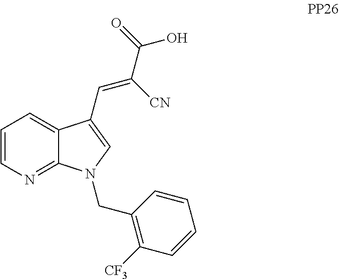 [SMILES: N#C/C(=C\c1cn(Cc2ccccc2C(F)(F)F)c2ncccc12)C(=O)O]

To the solution of compound 1 (1.0 equiv., 2.82 mmol, 332.8 mg) in dry DMF (5.6 mL) were added 2-(trifluoromethyl)benzyl bromide (1.2 equiv., 3.38 mmol, 808.1 mg) and KOH (1.2 equiv., 3.38 mmol, 189.6 mg) at 0° C. The reaction mixture was stirred at 21° C. for 2 h. After the reaction completion shown by TLC, water (5.6 mL) was added to the reaction vial. The reaction mixture was extracted by dichloromethane (14 mL×3). The combined organic layer was dried by sodium sulfate and concentrated. The residue was purified by flash column chromatography (hexanes/EtOAc=12:1) to provide the desired product, compound 2 (yield 89%, 690.3 mg) as a yellow solid.

POCl₃ (1 equiv., 2.29 mmol, 213.5 μL) was added dropwise to DMF (4.58 mL) at 0° C. under argon. After stirring for 10 min, a solution of compound 2 (1 equiv., 2.29 mmol, 632.2 mg) in DMF (4.58 mL) was added slowly with stirring. The mixture was kept at 21° C. overnight. The reaction was quenched by adding water (4.58 mL) at 0° C., then extracted with dichloromethane (13.74 mL×3). The combined organic layer was dried by sodium sulfate and concentrated. The residue was purified by flash column chromatography (hexanes/EtOAc=4:1) to provide the desired product, compound 3 (yield 79%, 552.1 mg) as a white solid.

To a solution of compound 3 (1.0 equiv., 1.77 mmol, 540.0 mg) in ethanol (7.0 mL) was added tert-butyl 2-cyanoacetate (1.3 equiv., 2.31 mmol, 289.9 μL) and L-proline (40 mol %, 0.71 mmol, 81.7 mg). The reaction was stirred at 21° C. for 12 h and a yellow solid precipitated gradually. After completion of the reaction, ice-cold water (7.0 mL) was added into the reaction vial. The solid was separated by Buchner funnel filtration and washed with water (7.0 mL×3) and dried to afford the desired product, compound 4 (yield 76%, 574.2 mg) as a white solid.

To a solution of compound 4 (1.0 equiv., 2.87 mmol, 1.23 g) was added 12 M aq. HCl (1025.8 equiv., 2942.4 mmol, 245.2 mL) at 0° C. The reaction mixture was stirred at 21° C. for 12 h. After the reaction was complete as shown by TLC, water (500 mL) was added at 0° C. The solid was filtered and washed with water (3×100 mL) then air dried. Finally, the product was dried in vacuo yielding the desired product, PP26 (yield 93%, 986.7 mg) as a light pink solid.

The following compounds were synthesized by a route similar to that described for PP26: PP27, PP28, PP31, PP32, PP33, PP34, PP35, PP36, PP37, PP38, PP39, PP40, PP51, PP52, PP53, PP54.

¹H NMR (500 MHz, DMSO-d₆) δ 13.50 (br s, 1H), 8.71 (s, 1H), 8.54-8.57 (m, 2H), 8.40 (dd, J=4.7, 1.5 Hz, 1H), 7.81 (d, J=7.6 Hz, 1H), 7.49-7.57 (m, 2H), 7.36 (dd, J=8.0, 4.7 Hz, 1H), 6.86 (d, J=7.6 Hz, 1H), 5.86 (s, 2H).

¹³C NMR (125 MHz, DMSO-d₆) δ 164.6, 147.9, 146.0, 145.6, 135.4 (2C), 133.7, 129.2, 128.9, 128.8, 126.8 (q, ³J_{C-F}=5.6 Hz), 126.6 (q, ²J_{C-F}=30.3 Hz), 124.8 (q, ¹J_{C-F}=274.0 Hz), 120.0, 119.1, 118.2, 108.7, 96.9, 45.4 (d, ⁴J_{C-F}=3.2 Hz).

### Example 2: (E)-2-Cyano-3-(1-(3-(trifluoromethyl)benzyl)-1H-pyrrolo[2,3-b]pyridin-3-yl)acrylic acid (PP27)

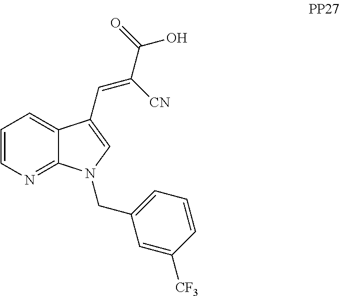 [SMILES: N#C/C(=C\c1cn(Cc2cccc(C(F)(F)F)c2)c2ncccc12)C(=O)O]

¹H NMR (500 MHz, DMSO-d₆) δ 13.49 (br s, 1H), 8.81 (s, 1H), 8.50-8.52 (m, 2H), 8.43 (dd, J=4.7, 1.5 Hz, 1H), 7.78 (s, 1H), 7.63-7.65 (m, 1H), 7.55-7.56 (2H), 7.34 (dd, J=8.0, 4.7 Hz, 1H), 5.76 (s, 2H).

¹³C NMR (125 MHz, DMSO-d₆) δ 164.7, 147.8, 146.1, 145.5, 138.8, 135.2, 132.2, 130.4, 129.8 (q, ²J_{C-F}=31.5 Hz), 129.1, 125.1 (q, ³J_{C-F}=3.8 Hz), 124.9 (q, ³J_{C-F}=3.9 Hz), 124.5 (q, ¹J_{C-F}=272.3 Hz), 120.0, 119.0, 118.3, 108.5, 96.8, 48.0.

### Example 3: (E)-2-Cyano-3-(1-(4-(trifluoromethyl)benzyl)-1H-pyrrolo[2,3-b]pyridin-3-yl)acrylic acid (PP28)

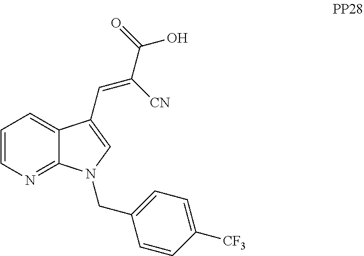 [SMILES: N#C/C(=C\c1cn(Cc2ccc(C(F)(F)F)cc2)c2ncccc12)C(=O)O]

¹H NMR (500 MHz, DMSO-d₆) δ 13.49 (s, 1H) 8.80 (s, 1H), 8.52-8.54 (m, 2H), 8.42 (dd, J=4.7, 1.5 Hz, 1H), 7.69 (d, J=7.9 Hz, 2H), 7.46-7.48 (m, 2H), 7.35 (dd, J=8.0, 4.7 Hz, 1H), 5.77 (s, 2H).

¹³C NMR (125 MHz, DMSO-d₆) δ 164.7, 147.8, 146.1, 145.6, 142.2, 135.3, 129.1, 128.8 (q, ²J_{C-F}=31.5 Hz), 128.6 (2C), 126.1 (q, ³J_{C-F}=3.8 Hz, 2C), 124.6 (q, ¹J_{C-F}=272.6 Hz), 120.0, 119.1, 118.3, 108.6, 96.7, 48.1.

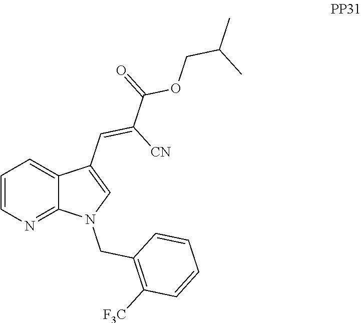 [SMILES: CC(C)COC(=O)/C(C#N)=C/c1cn(Cc2ccccc2C(F)(F)F)c2ncccc12]

### Example 3: Isobutyl (E)-2-cyano-3-(1-(2-(trifluoromethyl)benzyl)-1H-pyrrolo[2,3-b]pyridin-3-yl)acrylate (PP31)

¹H NMR (500 MHz, CDCl₃) δ 8.54 (s, 1H), 8.49 (s, 1H), 8.47 (dd, J=4.7, 1.3 Hz, 1H), 8.23 (dd, J=8.0, 1.4 Hz, 1H), 7.74 (dt, J=7.7, 3.7 Hz, 1H), 7.41 (dd, J=5.8, 3.4 Hz, 2H), 7.32 (dd, J=8.0, 4.7 Hz, 1H), 6.90 (dd, J=5.2, 3.6 Hz, 1H), 5.82 (s, 2H), 4.08 (d, J=6.6 Hz, 2H), 2.07 (hept, J=6.7 Hz, 1H), 1.01 (d, J=6.7 Hz, 6H).

¹³C NMR (125 MHz, CDCl₃) δ 163.48, 148.01, 145.53, 145.07, 134.38, 133.82, 132.52, 128.53, 128.21, 127.90 (q, ²J_{C-F}=32.4 Hz), 127.53, 126.43 (q, ³J_{C-F}=5.6 Hz), 124.22 (q, ¹J_{C-F}=273.6 Hz), 120.10, 118.76, 117.37, 109.03, 96.48, 72.04, 45.26 (d, ³J_{C-F}=3.2 Hz), 27.84, 19.02 (2C).

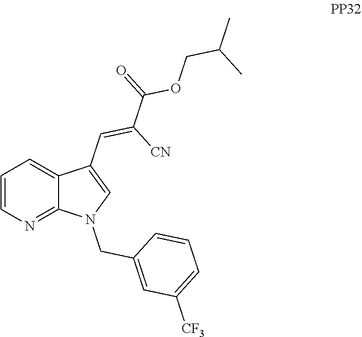 [SMILES: CC(C)COC(=O)/C(C#N)=C/c1cn(Cc2cccc(C(F)(F)F)c2)c2ncccc12]

### Example 4: Isobutyl (E)-2-cyano-3-(1-(3-(trifluoromethyl)benzyl)-1H-pyrrolo[2,3-b]pyridin-3-yl)acrylate (PP32)

¹H NMR (500 MHz, CDCl₃) δ 8.62 (s, 1H), 8.46-8.48 (m, 2H), 8.18 (dd, J=8.0, 1.5 Hz, 1H), 7.59 (s, 1H), 7.55-7.57 (m, 1H), 7.43-7.47 (m, 2H), 7.30 (dd, J=7.9, 4.7 Hz, 1H), 5.63 (s, 2H), 4.08 (d, J=6.7 Hz, 2H), 2.07 (hept, J=6.7 Hz, 1H), 1.01 (d, J=6.7 Hz, 6H).

¹³C NMR (125 MHz, CDCl₃) δ 163.4, 147.7, 145.5, 145.1, 136.9, 131.4 (q, ²J_{C-F}=32.5 Hz), 130.9, 130.9, 129.6, 127.5, 125.2 (q, ³J_{C-F}=3.7 Hz), 124.5 (q, ³J_{C-F}=3.8 Hz), 123.8 (q, ¹J_{C-F}=272.5 Hz), 120.3, 118.7, 117.5, 109.0, 96.4, 72.1, 48.6, 27.8, 19.0 (2C).

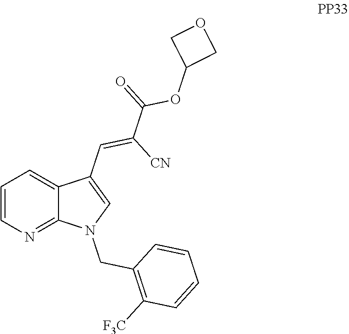 [SMILES: N#C/C(=C\c1cn(Cc2ccccc2C(F)(F)F)c2ncccc12)C(=O)OC1COC1]

### Example 5: Oxetan-3-yl (E)-2-cyano-3-(1-(2-(trifluoromethyl)benzyl)-1H-pyrrolo[2,3-b]pyridin-3-yl)acrylate (PP33)

¹H NMR (500 MHz, CDCl₃) δ 8.57 (s, 1H), 8.48-8.50 (m, 2H), 8.23 (dd, J=8.0, 1.5 Hz, 1H), 7.74-7.77 (m, 1H), 7.41-7.44 (m, 2H), 7.34 (dd, J=8.0, 4.7 Hz, 1H), 6.91-6.95 (m, 1H), 5.83 (s, 2H), 5.59-5.64 (m, 1H), 4.94-4.97 (m, 2H), 4.77-4.79 (m, 2H).

¹³C NMR (125 MHz, CDCl₃) δ 162.8, 148.1, 146.1, 145.7, 134.5, 134.2, 132.6, 128.6, 128.3, 127.9 (q, 2J_{C-F}=31.6 Hz), 127.6, 126.5 (q, ³J_{C-F}=5.7 Hz), 124.2 (q, ¹J_{C-F}=273.9 Hz), 120.1, 119.0, 117.1, 109.1, 95.0, 69.2 (3C), 45.4 (q, ⁴J_{C-F}=3.1 Hz).

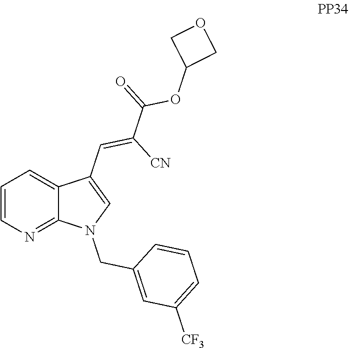 [SMILES: N#C/C(=C\c1cn(Cc2cccc(C(F)(F)F)c2)c2ncccc12)C(=O)OC1COC1]

### Example 6: Oxetan-3-yl (E)-2-cyano-3-(1-(3-(trifluoromethyl)benzyl)-1H-pyrrolo[2,3-b]pyridin-3-yl)acrylate (PP34)

¹H NMR (500 MHz, CDCl₃) δ 8.66 (s, 1H), 8.48-8.49 (m, 2H), 8.19 (dd, J=8.0, 1.5 Hz, 1H), 7.57-7.60 (m, 2H), 7.45-7.48 (m, 2H), 7.33 (dd, J=8.0, 4.7 Hz, 1H), 5.60-5.64 (m, 3H), 4.94-4.97 (m, 2H), 4.77-4.80 (m, 2H).

¹³C NMR (125 MHz, CDCl₃) δ 162.7, 147.8, 146.1, 145.7, 136.7, 133.8, 131.4 (q, ²J_{C-F}=32.6 Hz), 131.0, 129.6, 127.5, 125.3 (q, ³J_{C-F}=3.7 Hz), 124.6 (q, ³J_{C-F}=3.8 Hz), 123.7 (q, ¹J_{C-F}=272.6 Hz), 120.3, 119.0, 117.3, 109.0, 94.9, 69.2 (3C), 48.7.

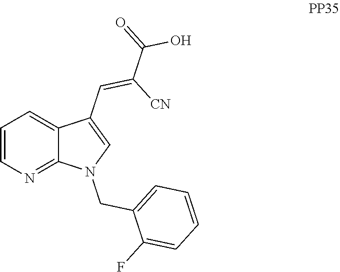 [SMILES: N#C/C(=C\c1cn(Cc2ccccc2F)c2ncccc12)C(=O)O]

### Example 7: (E)-2-cyano-3-(1-(2-fluorobenzyl)-1H-pyrrolo[2,3-b]pyridin-3-yl)acrylic acid (PP35)

¹H NMR (500 MHz, DMSO-d₆) δ 13.48 (br s, 1H), 8.72 (s, 1H), 8.50-8.52 (m, 2H), 8.42 (dd, J=4.6, 1.3 Hz, 1H), 7.33-7.38 (m, 2H), 7.23 (t, J=8.8 Hz, 2H), 7.13-7.16 (m, 1H), 5.69 (s, 2H).

¹³C NMR (125 MHz, DMSO-d₆) δ 164.7, 160.6 (d, ¹J_{C-F}=245.8 Hz), 147.8, 146.0, 145.5, 135.1, 130.8 (d, ³J_{C-F}=8.2 Hz), 130.7 (d, ³J_{C-F}=3.7 Hz), 129.0, 125.2 (d, ⁴J_{C-F}=3.5 Hz), 124.0 (d, ²J_{C-F}=14.7 Hz), 120.0, 119.0, 118.4, 116.1 (d, ²J_{C-F}=20.9 Hz), 108.4, 96.6, 42.9 (d, ³J_{C-F}=3.9 Hz).

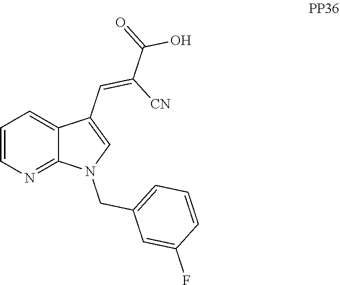 [SMILES: N#C/C(=C\c1cn(Cc2cccc(F)c2)c2ncccc12)C(=O)O]

### Example 8: (E)-2-cyano-3-(1-(3-fluorobenzyl)-1H-pyrrolo[2,3-b]pyridin-3-yl)acrylic acid (PP36)

¹H NMR (500 MHz, DMSO-d₆) δ 13.48 (br s, 1H), 8.76 (s, 1H), 8.50-8.52 (m, 2H), 8.43 (dd, J=4.7, 1.5 Hz, 1H), 7.33-7.39 (m, 2H), 7.09-7.17 (m, 3H), 5.67 (s, 2H).

¹³C NMR (125 MHz, DMSO-d₆) δ 164.7, 162.6 (d, ¹J_{C-F}=244.2 Hz), 147.8, 146.1, 145.5, 140.2 (d, ³J_{C-F}=7.3 Hz), 135.2, 131.3 (d, ³J_{C-F}=8.4 Hz), 129.1, 124.1 (d, ⁴J_{C-F}=2.7 Hz), 120.0, 119.0, 118.3, 115.1 (d, ²J_{C-F}=20.9 Hz), 114.9 (d, ²J_{C-F}=21.9 Hz), 108.5, 96.6, 48.0 (d, ⁴J_{C-F}=1.1 Hz).

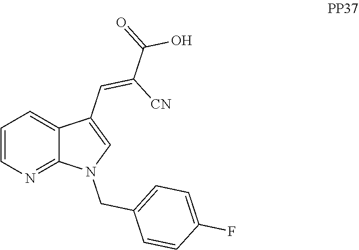 [SMILES: N#C/C(=C\c1cn(Cc2ccc(F)cc2)c2ncccc12)C(=O)O]

### Example 8: (E)-2-cyano-3-(1-(4-fluorobenzyl)-1H-pyrrolo[2,3-b]pyridin-3-yl)acrylic acid (PP37)

¹H NMR (500 MHz, DMSO-d₆) δ 8.74 (s, 1H), 8.49-8.51 (m, 2H), 8.43 (dd, J=4.7, 1.3 Hz, 1H), 7.39 (dd, J=8.6, 5.5 Hz, 2H), 7.34 (dd, J=8.0, 4.7 Hz, 1H), 7.14-7.17 (m, 2H), 5.63 (s, 2H).

¹³C NMR (125 MHz, DMSO-d₆) δ 164.7, 162.1 (d, ¹J_{C-F}=243.8 Hz), 147.8, 146.1, 145.5, 135.0, 133.6 (d, ⁴J_{C-F}=3.1 Hz), 130.4 (d, ³J_{C-F}=8.3 Hz, 2C), 129.0, 120.0, 119.0, 118.3, 116.0 (d, ²J_{C-F}=21.5 Hz, 2C), 108.4, 96.4, 47.8.

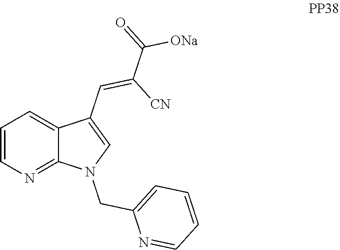

### Example 9: Sodium (E)-2-cyano-3-(1-(pyridin-2-ylmethyl)-1H-pyrrolo[2,3-b]pyridin-3-yl)acrylate (PP38)

¹H NMR (500 MHz, DMSO-d₆) δ 8.80 (s, 1H), 8.47-8.53 (m, 3H), 8.37 (dd, J=4.7, 1.5 Hz, 1H), 7.79 (td, J=7.7, 1.8 Hz, 1H), 7.29-7.34 (m, 3H), 5.77 (s, 2H).

¹³C NMR (125 MHz, DMSO-d₆) δ 164.7, 156.0, 149.5, 147.9, 146.1, 145.3, 138.0, 136.1, 128.9, 123.5, 122.4, 120.0, 118.9, 118.4, 108.3, 96.2, 50.0.

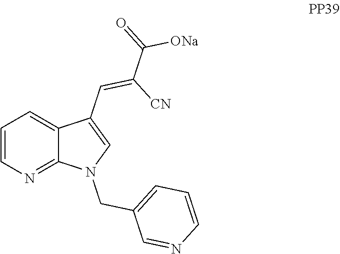

### Example 10: Sodium (E)-2-cyano-3-(1-(pyridin-3-ylmethyl)-1H-pyrrolo[2,3-b]pyridin-3-yl)acrylate (PP39)

¹H NMR (500 MHz, DMSO-d₆) δ 8.88 (d, J=1.9 Hz, 1H), 8.85 (s, 1H), 8.72 (dd, J=5.4, 1.3 Hz, 1H), 8.51-8.54 (m, 2H), 8.42 (dd, J=4.7, 1.5 Hz, 1H), 8.23 (dt, J=8.1, 1.6 Hz, 1H), 7.79 (dd, J=8.0, 5.4 Hz, 1H), 7.36 (dd, J=8.0, 4.7 Hz, 1H), 5.82 (s, 2H).

¹³C NMR (125 MHz, DMSO-d₆) δ 164.6, 147.7, 146.0, 145.5, 144.5, 144.7, 142.3, 135.7, 135.3, 129.2, 126.5, 120.17, 119.2, 118.2, 108.7, 97.0, 45.9.

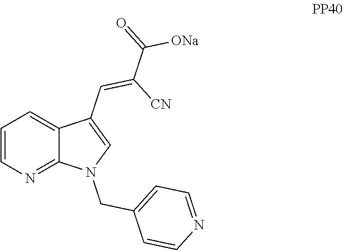

### Example 11: Sodium (E)-2-cyano-3-(1-(pyridin-4-ylmethyl)-1H-pyrrolo[2,3-b]pyridin-3-yl)acrylate (PP40)

¹H NMR (500 MHz, DMSO-d₆) δ 8.87 (s, 1H), 8.77 (d, J=6.6 Hz, 2H), 8.56 (d, J=9.2 Hz, 1H), 8.55 (s, 1H), 8.38 (d, J=4.7 Hz, 1H), 7.69 (d, J=6.3 Hz, 2H), 7.37 (dd, J=8.0, 4.7 Hz, 1H), 5.98 (s, 2H).

¹³C NMR (125 MHz, DMSO-d₆) δ 164.6, 156.0, 147.7, 145.9, 145.7, 143.8 (2C), 135.4, 129.3, 124.9 (2C), 120.1, 119.3, 118.1, 109.0, 97.4, 48.0.

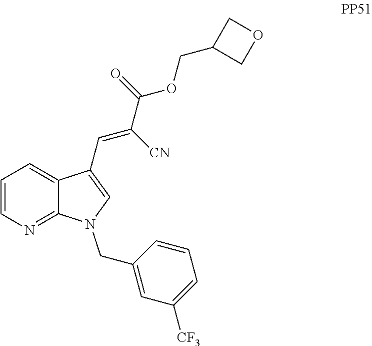 [SMILES: N#C/C(=C\c1cn(Cc2cccc(C(F)(F)F)c2)c2ncccc12)C(=O)OCC1COC1]

### Example 12: Oxetan-3-ylmethyl (E)-2-cyano-3-(1-(3-(trifluoromethyl)benzyl)-1H-pyrrolo[2,3-b]pyridin-3-yl)acrylate (PP51)

¹H NMR (500 MHz, CDCl₃) δ 8.61 (s, 1H), 8.48 (d, J=0.8 Hz, 1H), 8.47 (dd, J=4.7, 1.5 Hz, 1H), 8.17 (dd, J=7.9, 1.5 Hz, 1H), 7.59 (s, 1H), 7.54-7.56 (m, 1H), 7.44 (d, J=5.3 Hz, 2H), 7.30 (dd, J=8.0, 4.7 Hz, 1H), 5.62 (s, 2H), 4.85 (dd, J=7.8, 6.4 Hz, 2H), 4.51-4.55 (m, 4H), 3.36-3.45 (m, 1H).

¹³C NMR (125 MHz, CDCl₃) δ 163.4, 147.7, 145.6, 145.6, 136.8, 133.5, 131.4 (q, ²J_{C-F}=33.4 Hz), 131.0, 129.6, 127.5, 125.2 (q, ³J_{C-F}=3.8 Hz), 124.6 (q, ³J_{C-F}=3.8 Hz), 123.8 (q, ¹J_{C-F}=272.8 Hz), 120.3, 118.9, 117.4, 109.0, 95.6, 74.0 (2C), 66.8, 48.6, 34.2.

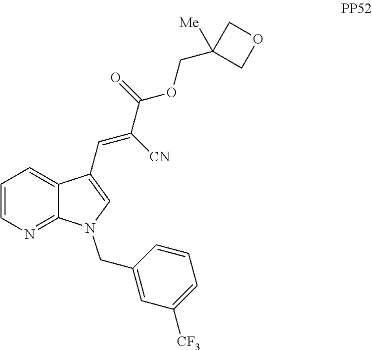 [SMILES: CC1(COC(=O)/C(C#N)=C/c2cn(Cc3cccc(C(F)(F)F)c3)c3ncccc23)COC1]

### Example 13: (3-methyloxetan-3-yl)methyl (E)-2-cyano-3-(1-(3-(trifluoromethyl)benzyl)-1H-pyrrolo[2,3-b]pyridin-3-yl)acrylate (PP52)

¹H NMR (500 MHz, CDCl₃) δ 8.62 (s, 1H), 8.50 (s, 1H), 8.47 (dd, J=4.7, 1.4 Hz, 1H), 8.17 (dd, J=8.0, 1.4 Hz, 1H), 7.59 (s, 1H), 7.55-7.56 (m, 1H), 7.44 (d, J=5.2 Hz, 2H), 7.30 (dd, J=7.9, 4.7 Hz, 1H), 5.63 (s, 2H), 4.57 (d, J=6.1 Hz, 2H), 4.44 (d, J=6.1 Hz, 2H), 4.41 (s, 2H), 1.41 (s, 3H).

¹³C NMR (125 MHz, CDCl₃) δ 163.4, 147.7, 145.6 (2C), 136.9, 133.5, 131.3 (q, ²J_{C-F}=31.5 Hz), 131.0, 129.6, 127.4, 125.2 (q, ³J_{C-F}=3.8 Hz), 124.6 (q, ³J_{C-F}=3.8 Hz), 123.8 (q, ¹J_{C-F}=272.8 Hz), 120.3, 118.9, 117.3, 109.0, 95.6, 79.5 (2C), 70.2, 48.6, 39.4, 21.0.

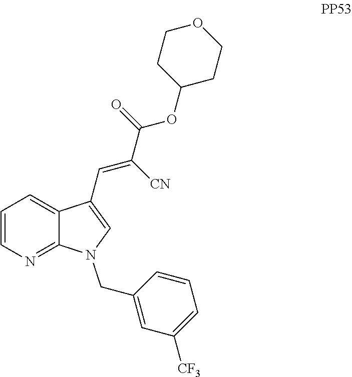 [SMILES: N#C/C(=C\c1cn(Cc2cccc(C(F)(F)F)c2)c2ncccc12)C(=O)OC1CCOCC1]

### Example 14: Tetrahydro-2H-pyran-4-yl (E)-2-cyano-3-(1-(3-(trifluoromethyl)benzyl)-1H-pyrrolo[2,3-b]pyridin-3-yl)acrylate (PP53)

¹H NMR (500 MHz, CDCl₃) δ 8.62 (s, 1H), 8.45-8.47 (m, 2H), 8.16 (dd, J=8.0, 1.5 Hz, 1H), 7.59 (s, 1H), 7.54-7.56 (m, 1H), 7.42-7.46 (m, 2H), 7.29 (dd, J=7.9, 4.7 Hz, 1H), 5.62 (s, 2H), 5.14 (hept, J=4.2 Hz, 1H), 3.96 (ddd, J=11.9, 6.0, 3.8 Hz, 2H), 3.59 (ddd, J=11.7, 8.3, 3.2 Hz, 2H), 1.97-2.03 (m, 2H), 1.80-1.85 (m, 2H).

¹³C NMR (125 MHz, CDCl₃) δ 162.7, 147.7, 145.5, 145.2, 136.9, 133.3, 131.4 (q, ²J_{C-F}=33.4 Hz), 130.9, 129.6, 127.4, 125.2 (q, ³J_{C-F}=3.8 Hz), 124.5 (q, ³J_{C-F}=3.8 Hz), 123.8 (q, ¹J_{C-F}=272.3 Hz), 120.3, 118.8, 117.5, 109.0, 96.4, 71.0, 65.1 (2C), 48.6, 31.6 (2C).

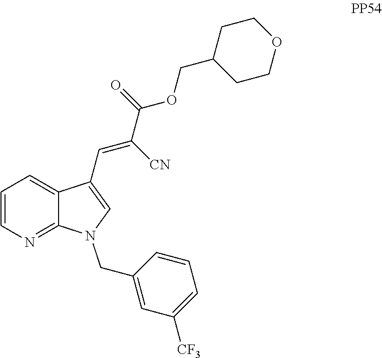 [SMILES: N#C/C(=C\c1cn(Cc2cccc(C(F)(F)F)c2)c2ncccc12)C(=O)OCC1CCOCC1]

### Example 15: (tetrahydro-2H-pyran-4-yl)methyl (E)-2-cyano-3-(1-(3-(trifluoromethyl)benzyl)-1H-pyrrolo[2,3-b]pyridin-3-yl)acrylate (PP54)

¹H NMR (500 MHz, CDCl₃) δ 8.61 (s, 1H), 8.45-8.47 (m, 2H), 8.17 (dd, J=8.0, 1.5 Hz, 1H), 7.59 (s, 1H), 7.54-7.56 (m, 1H), 7.44-7.45 (m, 2H), 7.30 (dd, J=7.9, 4.7 Hz, 1H), 5.62 (s, 2H), 4.15 (d, J=6.7 Hz, 2H), 3.99 (dd, J=11.3, 3.0 Hz, 2H), 3.41 (td, J=11.9, 2.1 Hz, 2H), 1.99-2.08 (m, 1H), 1.70 (dd, J=12.8, 1.8 Hz, 2H), 1.42 (qd, J=12.0, 4.5 Hz, 2H).

¹³C NMR (125 MHz, CDCl₃) δ 163.4, 147.72, 145.5, 145.3, 136.9, 133.3, 131.4 (q, ²J_{C-F}=33.4 Hz), 130.9, 129.6, 127.5, 125.2 (q, ³J_{C-F}=3.8 Hz), 124.5 (q, ³J_{C-F}=3.8 Hz), 123.8 (q, ¹J_{C-F}=272.8 Hz), 120.3, 118.8, 117.5, 109.0, 96.0, 70.1, 67.4 (2C), 48.6, 34.6, 29.4 (2C).

### Testing Methods

### Example 15

### Solubility Testing:

The solubility of the compounds prepared as described above was tested in a formulation containing about 25-75% polyethylene glycol, about 5-20% transcutol, about 5-50% ethanol, and about 5-10% DMSO. Results are detailed in the table below.

| Compound | | |
|---|---|---|
| ID | Conc. | Notes |
| JXL069 | 0.027% w/w | Stirred overnight at room temperature; dissolved |
| PP26 | 0.1% w/w | Stirred 1 hour at room temperature; dissolved |
| PP27 | 0.15% w/w | Stirred 1 hour at room temperature; dissolved |
| PP35 | 0.1% w/w | Stirred 24 hours at room temperature; failed to |
| | | dissolve |
| PP36 | 0.1% w/w | Stirred 24 hours at room temperature; failed to |
| | | dissolve |
| PP55 | 0.4% w/w | Stirred 1 hour at room temperature; dissolved. |
| | | Slightly opaque at 1.0% w/w |
| PP56 | 0.1% w/w | Stirred 24 hours at room temperature; failed to |
| | | dissolve |
| PP57 | 0.3% w/w | Stirred 1 hour at room temperature; dissolved |

### Example 16

### General Information

The study was designed to test Mitochondrial Pyruvate Carrier (MPC) by measuring respiration driven through Complex I of the electron transport chain using pyruvate as a substrate. Permeabilized HepG2 cells were acutely treated with test compounds in a nine-point dose-response. Permeabilizing the cells allowed for direct testing of pyruvate oxidation, which requires pyruvate transport into the mitochondria through the MPC. Oxygen consumption was evaluated using the XF96 platform by measuring oxygen consumption rates (OCR) in the presence of ADP to measure maximal ATP synthesis capacity (State 3) and in the presence of FCCP to determine maximal substrate oxidation (uncoupled respiration). UK5099, an MPC inhibitor, was used as a positive control to inhibit mitochondrial pyruvate oxidation.

### HepG2 Cells

HepG2 cells were cultured in the DMEM medium supplemented with 5.5 mM glucose, 4 mM glutamine, 1 mM pyruvate, and 10% FBS. HepG2 cells were plated in poly-d-lysine-coated XF96 microplates at 8,000 cells per well and maintained in a cell culture incubator overnight (37° C. incubator with 5% CO₂). On the day of the assay, test compounds were prepared in MAS buffer (70 mM sucrose, 220 mM mannitol, 5 mM potassium phosphate, 5 mM magnesium chloride, 1 mM EGTA, and 2 mM HEPES, pH 7.2 adjusted with KOH) containing 2× pyruvate (10 mM), malate (1 mM), ADP (8 mM), and recombinant, mutant perfringolysin O (PFO; XF PMP; Agilent Technologies; 10 nM) to permeabilize the plasma membrane. The cells were washed twice with MAS and then 75 μL of 2× compound, pyruvate, malate, ADP, and PFO was added to cells in 75 μL of MAS. The cells were incubated in a 37° C. incubator without CO₂ for 10 minutes before loading the plate into the XF96 Analyzer.

The injection ports of the XF96 Assay Cartridge were loaded with compounds that target the electron transport chain, which were injected during the assay. The assay included sequential injection of:
(a) The ATP Synthase inhibitor, oligomycin;
(b) The chemical uncoupler, FCCP, to release the control of mitochondrial ATP synthesis over respiration and determine maximal respiratory capacity under pyruvate; and
(c) Complex I inhibitor, rotenone, and Complex III inhibitor, antimycin A, to halt all mitochondrial respiration

No compound washout period occurred before injection of the compounds. The following table summarizes the order in which compounds were injected, the injection volume and concentration, as well as the final concentration of the compounds in the well to which the HepG2 cells were exposed.

| | | Injection | Injected | Final |
|---|---|---|---|---|
| Port | Compound | Volume | Concentration | Concentration |
| A | Oligomycin | 25 μL | 14 μM | 2 μM |
| B | FCCP | 25 μL | 8 μM | 1 μM |
| C | FCCP | 25 μL | 8 μM | 1.8 μM |
| D | Rot/Anti A | 25 μL | 20 μM | 2 μM |

Approximately 30 minutes before the end of the assay medium incubation, the XF96 Assay Cartridge was calibrated. Once the calibration process was completed, the microplate was placed into the instrument to begin the assay. The XF96 Extracellular Flux Analyzer protocol for HepG2 cells was as follows:

| Command | Time (minutes) | Port | Repeat |
|---|---|---|---|
| Calibrate | 30 | | |
| Mix | 2 | | 4 |
| Measue | 3 | | |
| Inject | A (oligomycin) | | |
| Mix | 2 | | 2 |
| Measure | 3 | | |
| Inject | B (FCCP) | | |
| Mix | 2 | | 2 |
| Measure | 3 | | |
| Inject | C (FCCP) | | |
| Mix | 2 | | 2 |
| Measure | 3 | | |
| Inject | D (Rotenone/Antimycin A) | | |
| Mix | 2 | | 3 |
| Measure | 3 | | |

End Program

### Normalization

Upon completion of each respirometry assay, the XF96 microplate was removed from the Seahorse Extracellular Flux Analyzer and fixed with 4% paraformaldehyde. After fixation, the cells were stained with 10 gig/mL Hoechst and cell number per well was assessed with an Operetta High-Content Imaging System. The respirometry well level data (pmoles O₂/min) was normalized per cell number (pmoles O₂/min/10³ cells) in each assay.

### Data Analysis

Each compound was run in duplicate and the average value of State 3 respiration and uncoupled respiration was calculated. Non-mitochondrial respiration (lowest value after injection of antimycin A and rotenone) was subtracted from all rates prior to calculating State 3 and uncoupled respiration. State 3 respiration was calculated as the average of the last 3 OCR measurements before injection of oligomycin in Port A. Uncoupled respiration was measured as the highest OCR after injection of FCCP. The IC₅₀ was calculated in Prism GraphPad by plotting the log concentration on the x-axis and OCR on the y-axis.

| Compound | Mean IC50 (nM) | Replicates |
|---|---|---|
| UK5099 | 33.9 | 3 |
| JXL069 | 51.2 | 3 |
| PP26 | 54.0 | 3 |
| PP27 | 36.3 | 3 |
| PP35 | 34 | 1 |
| PP36 | 26 | 1 |
| PP37 | 109 | 1 |
| PP38 | 183 | 1 |
| PP39 | 262 | 1 |
| PP40 | 411 | 1 |

### Example 17: Measurement of Esterase Activity of Selected Compounds in Human Skin Homogenate

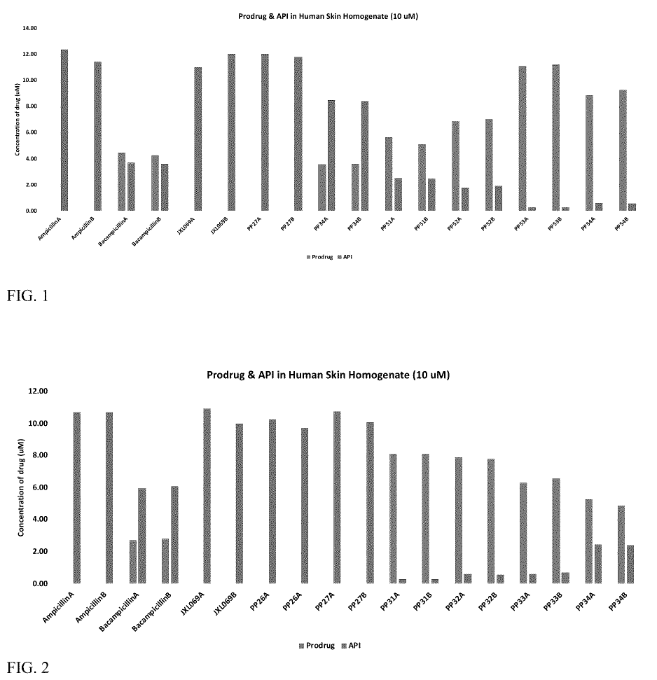

Samples of PP31, PP32, PP33, PP34, PP51, PP52, PP53, PP54, and references bacampicillin, ampicillin, JXL069, PP26, PP27 were prepared at a concentration of 10 μM, and incubated with homogenized human skin (0.5 mg/mL) in PBS (saline) solution (total volume of 1 mL) for 1 hour at 37° C. The samples were then quenched with UK5099 and analysis was performed using LCMS. Results are shown in FIGS. 1-2.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained. At the very least, and not as an attempt to limit the application of the doctrine of equivalents to the scope of the claims, each numerical parameter should at least be construed in light of the number of reported significant digits and by applying ordinary rounding techniques.

The terms “a,” “an,” “the” and similar referents used in the context of describing the invention (especially in the context of the following claims) are to be construed to cover both the singular and the plural, unless otherwise indicated herein or clearly contradicted by context. All methods described herein may be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of any claim. No language in the specification should be construed as indicating any non-claimed element essential to the practice of the invention.

Groupings of alternative elements or embodiments disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability.

Certain embodiments are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, the claims include all modifications and equivalents of the subject matter recited in the claims as permitted by applicable law. Moreover, any combination of the above-described elements in all possible variations thereof is contemplated unless otherwise indicated herein or otherwise clearly contradicted by context.

In closing, it is to be understood that the embodiments disclosed herein are illustrative of the principles of the claims. Other modifications that may be employed are within the scope of the claims. Thus, by way of example, but not of limitation, alternative embodiments may be utilized in accordance with the teachings herein. Accordingly, the claims are not limited to embodiments precisely as shown and described.

1-71. (canceled)

72. A method of growing hair, comprising: administering a compound to a mammal in need of hair growth, wherein the compound is represented by a formula: 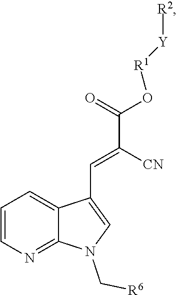
or a pharmaceutically acceptable salt thereof;
wherein R¹ is —, —S(═O)₂—, an optionally substituted C₁₋₁₂ hydrocarbon group or an optionally substituted heterocycle;
R² is H, an optionally substituted C₁₋₆ alkyl, an optionally substituted carbocycle, or an optionally substituted heterocycle;
Y is —, 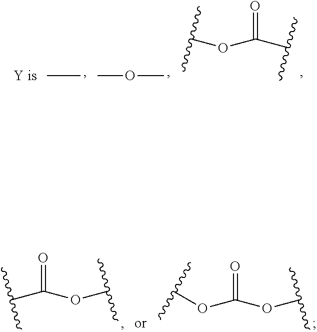
and
R⁶ is:

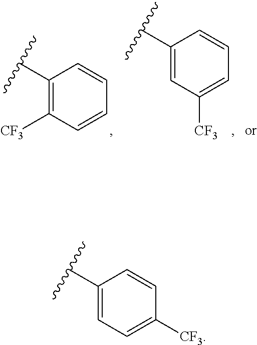 [SMILES: *c1ccccc1C(F)(F)F ; *c1cccc(C(F)(F)F)c1 ; *c1ccc(C(F)(F)F)cc1]

73. The method of claim 72, wherein R⁶ is:

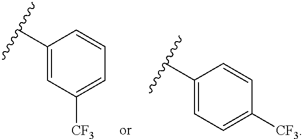 [SMILES: *c1cccc(C(F)(F)F)c1 ; *c1ccc(C(F)(F)F)cc1]

74. The method of claim 72, wherein R¹ is —, —CH₂—, an optionally substituted C₃₋₁₂ hydrocarbon group, or an optionally substituted heterocycle having a carbon atom directly attached to the O atom.

75. The method of claim 72, wherein R¹ is an optionally substituted C₁₋₁₂ alkyl.

76. The method of claim 72, wherein R¹ is an optionally substituted heterocycle.

77. The method of claim 72, wherein R¹ is an optionally substituted aryl.

78. The method of claim 72, wherein R¹ is an optionally substituted benzyl.

79. The method of claim 72, wherein R¹ is an optionally substituted oxetane, optionally substituted dihydrofuran, optionally substituted furan, optionally substituted furanone, optionally substituted tetrahydropyran, optionally substituted dihydropyran, optionally substituted pyran, optionally substituted tetrahydropyrone, optionally substituted dihydropyrone, optionally substituted thietane, optionally substituted tetrahydrothiophene, optionally substituted dihydrothiophene, optionally substituted thiophene, optionally substituted azetidine, optionally substituted pyrrolidine, optionally substituted pyrroline, optionally substituted pyrrole, optionally substituted piperidine, optionally substituted pyridine, optionally substituted oxazole, optionally substituted isoxazole, optionally substituted thiazole, optionally substituted isothiazole, optionally substituted pyrazolidine, optionally substituted imidazolidine, optionally substituted pyrazole, optionally substituted imidazole, optionally substituted tetrazole, or optionally substituted sulfolane.

80. The method of claim 72, wherein Y is —.

81. The method of claim 72, wherein Y is —O—.

82. The method of claim 72, wherein Y is

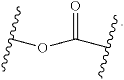 [SMILES: *OC(*)=O]

83. The method of claim 72, wherein Y is

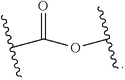 [SMILES: *OC(*)=O]

84. The method of claim 72, wherein Y is

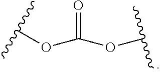 [SMILES: *OC(=O)O*]

85. The method of claim 72, wherein R² is H.

86. The method of claim 72, wherein the compound is 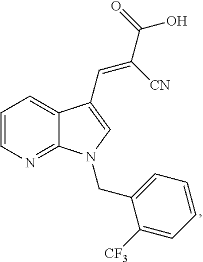 [SMILES: N#C/C(=C\c1cn(Cc2ccccc2C(F)(F)F)c2ncccc12)C(=O)O]
or a pharmaceutically acceptable salt thereof.

87. The method of claim 72, wherein the compound is 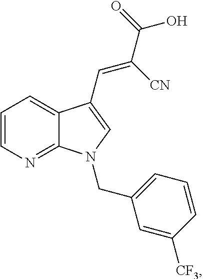 [SMILES: N#C/C(=C\c1cn(Cc2cccc(C(F)(F)F)c2)c2ncccc12)C(=O)O]
or a pharmaceutically acceptable salt thereof.

88. A compound of claim 72, wherein the compound is 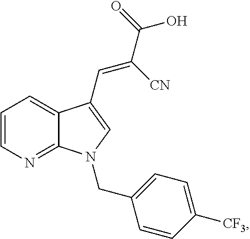 [SMILES: N#C/C(=C\c1cn(Cc2ccc(C(F)(F)F)cc2)c2ncccc12)C(=O)O]
or a pharmaceutically acceptable salt thereof.
